(19) United States
(12) Patent Application Publication
(10) Pub. No.: US 2018/0038865 A1
(43) Pub. Date: Feb. 8, 2018

(54) CD9 AS A THERAPEUTIC TARGET FOR HEMATOLOGIC MALIGNANCIES

(71) Applicant: The Chinese and Knowledge Transfer Services, Shatin (CN)

(72) Inventors: Kam Tong Leung, Shatin (CN); Chi Kong Li, Shatin (CN); Pak Cheung Ng, Shatin (CN); Karen Li, Shatin (CN); Ting Fan Leung, Shatin (CN); Yuen Yee Kathy Chan, Shatin (CN)

(21) Appl. No.: 15/653,325

(22) Filed: Jul. 18, 2017

Related U.S. Application Data
(60) Provisional application No. 62/370,575, filed on Aug. 3, 2016.

(51) Int. Cl. G01N 33/574; C12Q 1/68

(52) U.S. Cl. CPC G01N 33/57407; C12Q 1/6886; G01N 2800/50; G01N 2333/70596; C12Q 2600/118; C12Q 2600/158

(57) ABSTRACT

The present invention provides a method for detecting and treating hematologic malignancies including leukemia such as acute lymphoblastic leukemia (ALL) in a subject and a method for determining disease prognosis among leukemia patients by detecting CD9 expression. A kit and device useful for such methods are also provided. In addition, the present invention provides a composition for treating hematologic malignancies such as leukemia by suppressing CD9 expression or activity.

### RELATED APPLICATIONS

This application claims priority to U.S. Provisional Patent Application No. 62/370,575, filed on Aug. 3, 2016, the contents of which are hereby incorporated by reference in the entirety for all purposes.

### BACKGROUND OF THE INVENTION

Hematologic malignancies or blood cancers collectively are among the top six most common cancers worldwide, with about a million new diagnoses every year. In the US, blood cancer diagnosis accounts for about 10% of all new cancer cases, with a person receiving a diagnosis of a blood cancer every 3 minutes and a person dying from a blood cancer every 9 minutes.

Because of the high prevalence of hematologic cancers and the vital importance of early diagnosis on patients' life expectancy, there exists an urgent need for new and more effective methods to diagnose, monitor, and treat hematologic cancers. This invention fulfills this and other related needs.

### BRIEF SUMMARY OF THE INVENTION

The present inventors have identified CD9 as a novel therapeutic/prognostic marker for human hematologic malignancies. More specifically, the inventors observed in their studies that, compared with normal individuals, overexpression of CD9 is frequently found on the surface of lymphoblasts obtained from patients suffering from blood cancers, especially in acute lymphoblastic leukemia (ALL) patients. Such overexpression of CD9 also correlates with a poor survival rate and/or a high disease recurrence rate among patients. On the other hand, suppression of CD9 activity by a neutralizing antibody has been shown to potently inhibit the growth of cancerous lymphoblasts in patient-derived xenografts.

As such, in the first aspect, the present invention provides a method for assessing the risk for a blood cancer in a subject. The method includes these steps: (a) measuring CD9 level in a sample comprising lymphoblasts taken from the subject, (b) comparing the CD9 level obtained in step (a) with a standard control, and (c) determining the subject, who has an increased CD9 level compared with the standard control, as having an increased risk for a blood cancer. In some embodiments, the blood cancer is acute lymphoblastic leukemia (ALL). In some embodiments, the CD9 level is CD9 protein level. In other embodiments, the CD9 level is CD9 mRNA level. In some embodiments, step (a) of the method comprises an immunoassay using an antibody that specifically binds the CD9 protein. In some embodiments, step (a) comprises flow cytometry. In some embodiments, step (a) comprises a polynucleotide amplification reaction, such as a polymerase chain reaction (PCR) including a reverse transcriptase-PCR (RT-PCR). In some embodiments, step (a) comprises a polynucleotide hybridization assay, such as a Southern Blot analysis or Northern Blot analysis or an in situ hybridization assay.

In some cases, the method is practiced, after the subject is indicated as having an increased risk for blood cancer, to further include a repeated step (a) at a later time using the sample type of sample from the subject to measure the CD9 level. If an increase is detected in the CD9 level at the later time when the repeated step (a) is performed as compared to the amount from the original step (a), this indicates a heightened risk of blood cancer; on the other hand, if a decrease is detected when the repeated step (a) is performed, this indicates a lessened risk for blood cancer. In some embodiments, when the subject is indicated as having an increased risk for blood cancer, the method is practices to further comprise a step of administering to the subject an inhibitor of CD9, such as a neutralizing antibody against CD9 (e.g., ALB6 antibody), and optionally the patient is receiving chemotherapy at the same time (e.g., the patient is receiving vincristine treatment).

In a second aspect, the present invention provides a method for assessing, in a patient who has received a diagnosis of a blood cancer, the likelihood of recurrence of blood cancer or death due to the blood cancer. The method includes these steps: (a) measuring CD9 level in a sample comprising lymphoblasts taken from the patient, (b) comparing the CD9 level obtained in step (a) with CD9 level from another sample of the same type obtained from a second patient also suffering from the blood cancer and measured by step (a), and (c) determining the patient, who has a higher CD9 level compared with the CD9 level determined in the other sample of the same type obtained from a second patient and measured by step (a), as having an increased risk for recurrence of the blood cancer or death due to the blood cancer compared with the second patient. In some embodiments, the second patient has a CD9 level essentially the same as the CD9 level determined in another sample of the same type that is (1) obtained from a healthy individual who does not have the blood cancer or an elevated risk for developing the blood cancer; and (2) measured by step (a).

In some embodiments of this method, the blood cancer is ALL. In some embodiments, the CD9 level is CD9 protein level. In some embodiments, step (a) of the method comprises an immunoassay using an antibody that specifically binds the CD9 protein. In some embodiments, step (a) comprises flow cytometry. In some embodiments, the CD9 level is CD9 mRNA level. In some embodiments, step (a) comprises a polynucleotide amplification reaction including a polymerase chain reaction (PCR), e.g., a reverse transcriptase-PCR (RT-PCR). In some embodiments, step (a) comprises a polynucleotide hybridization reaction. In some embodiments, the patient being assessed for the likelihood of recurrence of blood cancer or death due to the blood cancer is one who has previously received treatment for the blood cancer. In some embodiments, the likelihood of recurrence of blood cancer or death due to the blood cancer is assessed for a time period of 5 years, 10 years, 15 years or longer after the patient first receives the blood cancer diagnosis.

In a third aspect, the present invention provides a kit for detecting or characterizing a blood cancer in a subject. The kit includes these components: (1) a standard control that provides an average amount of CD9 protein or CD9 mRNA in lymphoblasts; and (2) an agent that specifically and quantitatively identifies CD9 protein or CD9 mRNA. In some embodiments, the agent is an antibody that specifically binds the CD9 protein, or a polynucleotide probe that hybridizes with CD9 mRNA. The agent optionally comprises a detectable moiety for ease of detection. In some embodiments, the kit further comprises an instruction manual.

In a fourth aspect, the present invention provides a method for inhibiting growth of a CD9+ lymphoblast cell. The method comprises the step of contacting the cell with an effective amount of a neutralizing antibody against CD9 protein or a nucleic acid encoding a polynucleotide sequence that is complementary to at least a segment of CD9 mRNA and suppresses CD9 mRNA expression. In some embodiments, the nucleic acid contains a promoter operably linked to the coding sequence for the polynucleotide complementary to at least a segment of CD9 mRNA. In some embodiments, the promoter is a lymphoblast-specific promoter. In some embodiments, the nucleic acid encodes an antisense RNA, miRNA, or siRNA. In some embodiments, the CD9+ lymphoblast cell is within a patient's body. In some embodiments, the patient is concurrently or sequentially receiving chemotherapy (e.g., vincristine treatment) and a neutralizing antibody against CD9 (e.g., ALB6 antibody).

In a related aspect, the present invention provides use of a CD9 inhibitor for manufacturing a medicament for treating blood cancers, especially leukemias such as ALL. The CD9 inhibitor, which may suppress CD9 level by reducing CD9 mRNA expression level, CD9 protein expression level, or CD9 protein activity, can be formulated with one or more physiologically acceptable excipients for administration to a patient who has been diagnosed with a blood cancer. The inhibitor may be a polynucleotide, such as an antisense RNA, miRNA, or siRNA targeting the CD9 mRNA, or a polypeptide, such as a neutralizing antibody against the CD9 protein (e.g., antibody ALB6). Optionally, medicament comprises the CD9 inhibitor and a second therapeutic agent for treating blood malignancies (e.g., vincristine).

### BRIEF DESCRIPTION OF THE DRAWINGS

FIGS. 1A-1D: Association of CD9 expression with survival outcomes in pediatric B-ALL patients. (FIGS. 1A-1D) Kaplan-Meier analyses of overall survival (OS, left panels) and relapse-free survival (RFS, right panels) in patients with CD9⁺ or CD9⁻ blasts at the time of diagnosis. (FIG. 1A) all patients of the study cohort (n=118), (FIG. 1B) high-risk patients (n=22), (FIG. 1C) intermediate-risk patients (n=59), (FIG. 1D) standard-risk patients (n=37). The 5-year OS, RFS rates and P values are indicated. Statistics: log-rank test.

FIGS. 2A-2C: Effects of CD9 antibody on B-ALL homing and engraftment. (FIGS. 2A-2C) 697 cells were pre-exposed to IgG or anti-CD9 (ALB6, 10 μg/mL) for 4 hours, and transplanted into sublethally irradiated NOD/SCID mice. Mice were harvested at (FIG. 2A) 20 hours post-transplant for analysis of leukemic cell homing, and at (FIGS. 2B and 2C) 14-20 days post-transplant for analysis of engraftment in different organs by flow cytometric detection of human CD45⁺CD19⁺ cells. (FIG. 2C) Mice were prior treated with anti-CD122 (200 μg/mouse) after irradiation to deplete residual NK cells. (FIGS. 2A and 2B) 5 mice/group, (FIG. 2C) 4 mice/group. Data are presented as (FIG. 2A) mean numbers of detected CD45⁺ CD19⁺ cells in 10⁶ of acquired cells and (FIGS. 2B and 2C) mean percentages of CD45⁺CD19⁺ cells in 10⁵ of acquired cells. Statistics: two-tailed, unpaired Student's t-test. **P<0.01, ***P<0.001, N.S. Not significant.

FIGS. 3A-3C: Effects of CD9 antibody treatment on leukemic progression of B-ALL cell line-xenografted mice. NOD/SCID mice were transplanted with B-ALL cell lines (FIG. 3A) 697 (TCF3-PBX1⁺, intermediate-risk), (FIG. 3B) RS4;11 (MLL-AF4⁺, high-risk) or (FIG. 3C) Reh (ETV6-RUNX1⁺, standard-risk) and treated with IgG or anti-CD9 (ALB6, 20 μg/mouse) twice a week for 2 weeks. (i) Expression of CD9 on B-ALL cell lines at the time of transplantation. (ii) Mice were sacrificed at (FIG. 3A) 14-17 days, (FIG. 3B) 38-41 days and (FIG. 3C) 22-30 days post-transplant for analysis of leukemic burden. (iii) Survival analysis of mice treated with IgG or anti-CD9. (A) 4-6 mice/group, (B) 4-5 mice/group, (C) 5-7 mice/group. Statistics: (ii) two-tailed, unpaired Student's t-test, (iii) log-rank test. *P<0.05, **P<0.01, ***P<0.001, N.S. Not significant.

FIGS. 4A-4D: Effects of CD9 antibody treatment on mice xenografted with patient B-ALL blasts. NOD/SCID mice were transplanted with B-ALL samples from 4 different patients: (FIG. 4A) TCF3-PBX1⁺ (intermediate-risk), (FIG. 4B) normal karyotype (intermediate-risk), (FIG. 4C) MLL-AF4⁺ (high-risk), (FIG. 4D) relapsed/refractory (high-risk). Mice were treated with IgG or anti-CD9 (ALB6, 20 μg/mouse) twice a week for 2 weeks. (i) Expression of CD9 on patient B-ALL blasts at transplantation. (ii) Comparison of leukemic burden at (FIG. 4A) 44-47 days, (FIG. 4B) 32-35 days, (FIG. 4C) 45-49 days, (FIG. 4D) 44-48 days post-transplant. (iii) Survival analysis. (FIGS. 4A, 4B and 4D) 4-5 mice/group, (FIG. 4C) 5 mice/group. Statistics: (ii) two-tailed, unpaired Student's t-test, (iii) log-rank test. **P<0.01, ***P<0.001, N.S. Not significant.

FIGS. 5A-5C: In vitro and in vivo effects of CD9 antibody on chemosensitivity of B-ALL cells. (FIG. 4A) B-ALL cell lines were treated with vincristine (VCR, 12.5 nM) for 3 days in monoculture or stromal co-culture. Apoptosis was monitored by Annexin V/7-AAD staining (n=4). (FIG. 4B) 697 cells were treated with VCR (12.5 nM) in stromal co-cultures for 3 days, in the presence of control IgG or anti-CD9 (ALB6, 10 μg/mL), and measured for apoptosis (n=4). (FIG. 4C) NOD/SCID mice were transplanted with 697 cells and treated with IgG alone, anti-CD9 alone (ALB6, 20 μg/mouse), IgG+VCR (0.8 mg/kg) or anti-CD9 + VCR. Antibodies and VCR were given twice a week for 2 weeks. Animal survival was analyzed (4-5 mice/group). Data are means±SEM. Statistics: (A and B) two-tailed, paired Student's t-test, (FIG. 4C) log-rank test. *P<0.05, **P<0.01.

FIGS. 6A-6G: Roles of CD9 in migration, adhesion and integrin affinity of B-ALL cells. (FIG. 6A) Migration of B-ALL cell lines to a SDF-1 gradient (100 ng/mL, n=3). (FIG. 6B) B-ALL cell lines were treated with IgG or anti-CD9 (ALB6, 10 μg/mL) for 4 hours, and subjected to migration assay (n=3). (FIG. 6C) Adhesion of B-ALL cell lines to BM-derived stromal cells (n=3). (FIG. 6D) B-ALL cell lines were exposed to IgG or anti-CD9 (ALB6, 10 μg/mL), and subjected to stromal adhesion assay (n=3). (FIG. 6E) Affinity of B-ALL cells to soluble VCAM-1 (10 μg/mL, n=4). (FIG. 6F) 697 cells were transduced with lentivirus encoding the CD9 or control sgRNA, sorted for GFP⁺ cells, and assessed for VCAM-1 binding level (n=3). (FIG. 6G) Lysates from B-ALL cell lines were immunoprecipitated with control IgG or anti-CD9 (MM2/57, 10 μg) and probed for integrins α4, β1 and CD9. Levels of these proteins in input lysates are shown in the right panel. Results are representative of 3 independent experiments. Data are presented as means±SEM. Statistics: (FIGS. 6A-6F) two-tailed, paired Student's t-test. *P<0.05, **P<0.01, ***P<0.001.

FIGS. 7A-7C: Effects of CD9 antibody on apoptosis, phagocytic signals and expression of adhesion molecules. 697 cells were treated with IgG or anti-CD9 (ALB6, 10 μg/mL) for 4 hours and monitored for (FIG. 7A) apoptosis, (FIG. 7B) alteration of pro-(calreticulin) and anti-phagocytic signals (CD47) and (FIG. 7C) changes in expression of adhesion molecules CXCR4, integrins α4 and β1 (n=3). Data are presented as means±SEM. Statistics: two-tailed, paired Student's t-test. N.S. Not significant.

FIG. 8: Effects of CD9 antibodies on engraftment of B-ALL cells. 697 cells were pre-exposed to IgG or anti-CD9 (M-L13 or MM2/57, 10 μg/mL) for 4 hours, and transplanted into sublethally irradiated NOD/SCID mice. Mice were harvested at 15-17 days post-transplant for analysis of leukemic cell engraftment (4 mice/group). Statistics: two-tailed, unpaired Student's t-test. *P<0.05, ***P<0.001.

FIG. 9: Expression of adhesion molecules on B-ALL cell lines. Flow cytometric analysis of cell surface CXCR4, integrins α4 and β1 expressions on B-ALL cell lines. Results are representatives of 3 independent experiments.

### DEFINITIONS

The term “CD9 gene” or “CD9 protein,” as used herein, refers to a member of the transmembrane 4 superfamily, also known as the tetraspanin family, a glycoprotein that is expressed on the surface of various cells and includes four hydrophobic transmembrane domains. Also encompassed in the term are any naturally occurring variants or mutants, interspecies homologs or orthologs, or man-made variants of human CD9 gene or CD9 protein. The cDNA sequence encoding a human wild-type CD9 mRNA is set forth in GenBank Accession No. NM_001769.3 (provided herein as SEQ ID NO:1), which translate to a 228-amino acid CD9 protein (GenBank Accession No. NP 001760.1, provided herein as SEQ ID NO:2). A CD9 protein within the meaning of this application typically has at least 80%, 85%, 90%, or 95% or higher sequence identity to the human wild-type CD9 protein.

In this disclosure the terms “hematologic malignancy” and “blood cancer” have the same meaning and generically refer to a cancer caused by abnormal hyper-proliferation of one group of blood cells. “Hematologic malignancies” include these main types: leukemia, lymphoma, and myeloma. Leukemia involves an abnormal number of immature white blood cells being produced in the bone marrow, which then overwhelms the bone marrow and hinders it normal functions. There are many types of leukemia: acute myeloid leukemia (AML), acute lymphoblastic leukemia (ALL), chronic myeloid leukemia (CML), and chronic lymphocytic leukemia (CLL) are relatively common, whereas the less common types include acute promyelocytic leukemia (APL), hairy cell leukemia (HCL), large granular lymphocytic leukemia (LGL), t-cell acute lymphoblastic leukaemia (T-ALL), chronic myelomonocytic leukemia (CMML). Lymphoma involves over-proliferation of lymphocytes in lymphatic tissues such as lymph nodes. The two main types of lymphoma are Hodgkin lymphoma and non-Hodgkin lymphoma. Myeloma, also known as multiple myeloma, involves unusually large numbers of abnormal plasma cells clogging up a patient's bone marrow and interfering with its normal functions. Hematological malignancies may derive from either of the two major blood cell lineages: myeloid cell line (which normally produces granulocytes, erythrocytes, thrombocytes, macrophages and mast cells) and lymphoid cell line (which normally produced B, T, NK and plasma cells). Lymphomas, lymphocytic leukemias, and myelomas are from the lymphoid line, while acute and chronic myelogenous leukemias are myeloid in origin.

In this disclosure, the term “lymphoblast” or “blast” is used to refer to a naive or immature form of a precursor cell of the blood lineage that occurs upon activation by an antigen (from antigen-presenting cells), exhibiting an increased volume of nucleus and cytoplasm as well as new mRNA and protein synthesis. The blast is capable of dividing and proliferating, and ultimately differentiating into mature blood cells, or effector cells such as B cells and T cells.

In this disclosure the term “or” is generally employed in its sense including “and/or” unless the content clearly dictates otherwise.

As used herein, the term “gene expression” is used to refer to the transcription of a DNA to form an RNA molecule encoding a particular protein (e.g., human CD9 protein) or the translation of a protein encoded by a polynucleotide sequence. In other words, both mRNA level and protein level encoded by a gene of interest (e.g., human CD9 gene) are encompassed by the term “gene expression level” in this disclosure.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., serum, plasma, platelets, red blood cells, white blood cells, and the like), sputum or saliva, lymph and tongue tissue, cultured cells, e.g., primary cultures, explants, and transformed cells, stool, urine, colon biopsy tissue etc. A biological sample is typically obtained from a eukaryotic organism, which may be a mammal, may be a primate and may be a human subject.

In this disclosure the term “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., whole blood, blood cells such as red or white blood cells, plasma/serum, lymph nodes, liver, bone marrow, spleen, etc.) among other factors. Representative biopsy techniques include, but are not limited to, excisional biopsy, incisional biopsy, needle biopsy, surgical biopsy, and bone marrow biopsy and may comprise blood drawing. A wide range of biopsy techniques are well known to those skilled in the art who will choose between them and implement them with minimal experimentation.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamine) containing restriction-digested genomic DNA, is not an “isolated” nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO01/12654, which may improve the stability (e.g., half-life), bioavailability, and other characteristics of a polypeptide comprising one or more of such D-amino acids. In some cases, one or more, and potentially all of the amino acids of a therapeutic polypeptide have D-chirality.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

As used in herein, the terms “identical” and percent “identity,” in the context of describing two or more polynucleotide or amino acid sequences, refer to two or more sequences or subsequences that are the same or have a specified percentage of amino acid residues or nucleotides that are the same (for example, a variant CD9 protein used in the method of this invention (e.g., for treating a blood cancer) has at least 80% sequence identity, preferably 85%, 90%, 91%, 92%, 93, 94%, 95%, 96%, 97%, 98%, 99%, or 100% identity, to a reference sequence, e.g., a wild-type human CD9 protein), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in lx SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

The phrase “specifically binds,” when used in the context of describing a binding relationship of a particular molecule (e.g., an anti-CD9 antibody) to a protein or peptide (e.g., a human CD9 protein), refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used in this application, an “increase” or a “decrease” refers to a detectable positive or negative change in quantity from a comparison control, e.g., an established standard control (such as an average expression level of CD9 mRNA or CD9 protein found in non-cancerous B cells obtained from healthy subjects). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term “inhibiting” or “inhibition,” as used herein, refers to any detectable negative effect on a target biological process, such as cellular signal transduction, cell proliferation, tumorigenicity, metastatic potential, and recurrence of a disease/condition. Typically, an inhibition is reflected in a decrease of at least 10%, 20%, 30%, 40%, or 50% in target process (e.g., expression of CD9 at either mRNA level or protein level) upon application of an inhibitor, when compared to a control where the inhibitor is not applied.

A “polynucleotide hybridization method” as used herein refers to a method for detecting the presence and/or quantity of a pre-determined polynucleotide sequence based on its ability to form Watson-Crick base-pairing, under appropriate hybridization conditions, with a polynucleotide probe of a known sequence. Examples of such hybridization methods include Southern blot, Northern blot, and in situ hybridization.

“Primers” as used herein refer to oligonucleotides that can be used in an amplification method, such as a polymerase chain reaction (PCR), to amplify a nucleotide sequence based on the polynucleotide sequence corresponding to a gene of interest, e.g., the cDNA or genomic sequence for human CD9 or a portion thereof. Typically at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art. In this disclosure the term “primer pair” means a pair of primers that hybridize to opposite strands a target DNA molecule or to regions of the target DNA which flank a nucleotide sequence to be amplified. In this disclosure the term “primer site”, means the area of the target DNA or other nucleic acid to which a primer hybridizes.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a probe or a molecule with defined binding characteristics (e.g., a polypeptide with a known binding specificity or a polynucleotide), so as to allow the presence of the probe (and therefore its binding target) to be readily detectable.

“Standard control” as used herein refers to a predetermined amount or concentration of a polynucleotide sequence or polypeptide, e.g., CD9 mRNA or protein, that is present in an established normal disease-free tissue sample, e.g., a lymphoblast sample obtained from the bone marrow of a healthy person not suffering from any hematopoietic disorder especially malignancy. The standard control value is suitable for the use of a method of the present invention, to serve as a basis for comparing the amount of CD9 mRNA or protein that is present in a test sample. An established sample serving as a standard control provides an average amount of CD9 mRNA or CD9 protein that is typical for a normal, non-cancerous lymphoblast sample taken from an average, healthy human without any hematopoietic malignancy especially leukemia as conventionally defined, preferably without any increased risk of developing the disease. A standard control value may vary depending on the nature of the sample, the manner by which it has been processed, as well as other factors such as the gender, age, ethnicity of the subjects based on whom such a control value is established.

The term “average,” as used in the context of describing a human who is healthy, free of any blood disease (especially blood cancer) as conventionally defined, refers to certain characteristics, especially the amount of CD9 mRNA or protein, found in the person's blood sample, e.g., lymphoblast sample, that are representative of a randomly selected group of healthy humans who are free of any hematopoietic disorders (especially malignancies such as leukemia) and free of known risk of developing the diseases. This selected group should comprise a sufficient number of humans such that the average amount of CD9 mRNA or protein in the B cells among these individuals reflects, with reasonable accuracy, the corresponding amount of CD9 mRNA/protein in the general population of healthy humans. In addition, the selected group of humans generally have a similar age to that of a subject whose lymphocyte sample is tested for indication of blood cancers. Moreover, other factors such as gender, ethnicity, medical history are also considered and preferably closely matching between the profiles of the test subject and the selected group of individuals establishing the “average” value.

The term “amount” as used in this application refers to the quantity of a polynucleotide of interest or a polypeptide of interest, e.g., human CD9 mRNA or protein, present in a sample. Such quantity may be expressed in the absolute terms, i.e., the total quantity of the polynucleotide or polypeptide in the sample, or in the relative terms, i.e., the concentration of the polynucleotide or polypeptide in the sample.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an polynucleotide encoding a CD9 antisense RNA is the amount of said polynucleotide to achieve a decreased level of CD9 mRNA or protein expression or biological activity, such that the symptoms, severity, and/or recurrence change of a blood cancer are reduced, reversed, eliminated, prevented, or delayed of the onset in a patient who has been given the polynucleotide for therapeutic purposes. An amount adequate to accomplish this is defined as the “therapeutically effective dose.” The dosing range varies with the nature of the therapeutic agent being administered and other factors such as the route of administration and the severity of a patient's condition.

The term “subject” or “subject in need of treatment,” as used herein, includes individuals who seek medical attention due to risk of, or actual suffering from, a blood cancer such as leukemia. Subjects also include individuals currently undergoing therapy that seek manipulation of the therapeutic regimen. Subjects or individuals in need of treatment include those that demonstrate symptoms of a blood cancer or are at risk of suffering from a blood cancer or its symptoms. For example, a subject in need of treatment includes individuals with a genetic predisposition or family history for leukemia cancer, those that have suffered relevant symptoms in the past, those that have been exposed to a triggering substance or event, as well as those suffering from chronic or acute symptoms of the condition. A “subject in need of treatment” may be at any age of life, although pediatric patients are disproportionally affected in certain forms of leukemia such as ALL.

“Inhibitors,” “activators,” and “modulators” of CD9 protein are used to refer to inhibitory, activating, or modulating molecules, respectively, identified using in vitro and in vivo assays for CD9 protein binding or signaling, e.g., ligands, agonists, antagonists, and their homologs and mimetics. The term “modulator” includes inhibitors and activators. Inhibitors are agents that, e.g., partially or totally block binding, decrease, prevent, delay activation, inactivate, desensitize, or down regulate the activity of CD9 protein. In some cases, the inhibitor directly or indirectly binds to CD9 protein, such as a neutralizing antibody. Inhibitors, as used herein, are synonymous with inactivators and antagonists. Activators are agents that, e.g., stimulate, increase, facilitate, enhance activation, sensitize or up regulate the activity of CD9 protein. Modulators include CD9 protein ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.

### DETAILED DESCRIPTION OF THE INVENTION

### I. Introduction

Despite major advances in risk-directed multi-agent chemotherapy, specific subtypes of high-risk and relapsed/refractory pediatric B-precursor acute lymphoblastic leukemia (B-ALL) remain associated with poor clinical outcomes, highlighting the need for development of novel and effective therapeutic strategies. In a cohort of pediatric B-ALL cases, the present inventors identified that the cell surface expression of tetraspanin protein CD9, particularly in high-risk patients, was significantly associated with inferior survival outcomes. Targeting CD9 with a neutralizing antibody substantially reduced leukemic burden and prolonged survival of NOD/SCID mice transplanted with cytogenetically diverse B-ALL cell lines and patient-derived leukemic blasts. Importantly, xenografts of relapsed/refractory B-ALL and those with high-risk features were sensitive to CD9 antibody treatment. Further, CD9 blockade significantly increased sensitivity of leukemic cells to chemotherapy in stromal co-culture and animal models. Mechanistic studies revealed that CD9 was functionally involved in leukemic cell migration and stromal adhesion by modulating the affinity of integrin very late antigen-4. These observations collectively indicate that CD9 blockade, in adjunct to chemotherapy, can be a promising strategy for treatment of high-risk and relapsed/refractory pediatric B-ALL, possibly mediated by disruption of leukemia-stroma interaction in the bone marrow microenvironment.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of the human CD9 gene, and synthetic oligonucleotides (e.g., primers) can be verified using, e.g., the chain termination method for double-stranded templates of Wallace et al., Gene 16:21-26 (1981).

III. Acquisition of Samples and Analysis of CD9 mRNA

The present invention relates to measuring the amount of CD9 mRNA found in a person's lymphoblasts as a means to detect the presence, to assess the risk of developing, and/or to monitor the progression or treatment efficacy of a blood cancer, such as leukemia, including assessing the likelihood of disease recurrence or patient survival within the next given time period. Thus, the first steps of practicing this invention are to obtain a sample comprising lymphoblasts from a test subject and extract mRNA from the lymphoblasts.

A. Acquisition and Preparation of Samples

A blood or bone marrow sample is obtained from a person to be tested or monitored for blood cancer using a method of the present invention. Collection of a peripheral blood or bone marrow sample from an individual is performed in accordance with the standard protocol hospitals or clinics generally follow. An appropriate amount of blood or bone marrow is collected and may be stored according to standard procedures prior to further preparation.

The analysis of CD9 mRNA found in a patient's sample according to the present invention may be performed using, e.g., lymphoblast cells cultured from either a blood sample or a bone marrow sample. The methods for preparing lymphoblast cell samples for nucleic acid extraction are well known among those of skill in the art. For example, a subject's lymphoblast sample should be first treated to disrupt cellular membrane so as to release nucleic acids contained within the cells.

B. Extraction and Quantitation of RNA

Methods for extracting RNA from a biological sample are well known and routinely practiced in the art of molecular biology (e.g., described by Sambrook and Russell, Molecular Cloning: A Laboratory Manual 3d ed., 2001). The general methods of mRNA preparation can be followed, see, e.g., Sambrook and Russell, supra, using various commercially available reagents or kits, such as Trizol reagent (Invitrogen, Carlsbad, Calif.), Oligotex Direct mRNA Kits (Qiagen, Valencia, Calif.), RNeasy Mini Kits (Qiagen, Hilden, Germany), and PolyATtract® Series 9600™ (Promega, Madison, Wis.). Combinations of more than one of these methods may also be used. It is essential that all contaminating DNA be eliminated from the RNA preparations. Thus, careful handling of the samples, thorough treatment with DNase, and proper negative controls in the amplification and quantification steps should be used.

1. PCR-Based Quantitative Determination of mRNA Level

Once mRNA is extracted from a sample, the amount of human CD9 mRNA may be quantified. The preferred method for determining the mRNA level is an amplification-based method, e.g., by polymerase chain reaction (PCR), especially reverse transcription-polymerase chain reaction (RT-PCR) for mRNA quantitative analysis.

To quantitatively measure CD9 mRNA level, the mRNA must be first reverse transcribed. Prior to the amplification step, a DNA copy (cDNA) of the human CD9 mRNA must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp.401-406; Persing et al., eds., Mayo Foundation, Rochester, Minn., 1993; Egger et al., J. Clin. Microbiol. 33:1442-1447, 1995; and U.S. Pat. No. 5,075,212.

The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

Although PCR amplification of the target mRNA is typically used in practicing the present invention, one of skill in the art will recognize, however, that amplification of these mRNA species in a sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to quantitatively determining the amount of mRNA in the sample. For a review of branched-DNA signal amplification for direct quantitation of nucleic acid sequences in clinical samples, see Nolte, Adv. Clin. Chem. 33:201-235, 1998.

2. Other Quantitative Methods

The CD9 mRNA can also be detected using other standard techniques, well known to those of skill in the art. Although the detection step is typically preceded by an amplification step, amplification is not required in the methods of the invention. For instance, the mRNA may be identified by size fractionation (e.g., gel electrophoresis), whether or not proceeded by an amplification step. After running a sample in an agarose or polyacrylamide gel and labeling with ethidium bromide according to well-known techniques (see, e.g., Sambrook and Russell, supra), the presence of a band of the same size as the standard comparison is an indication of the presence of a target mRNA, the amount of which may then be compared to the control based on the intensity of the band. Alternatively, oligonucleotide probes specific to CD9 mRNA can be used to detect the presence of such mRNA species and indicate the amount of mRNA in comparison to the standard comparison, based on the intensity of signal imparted by the probe.

Sequence-specific probe hybridization is a well-known method of detecting a particular nucleic acid comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch.

A number of hybridization formats well-known in the art, including but not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

The hybridization complexes are detected according to well-known techniques. Nucleic acid probes capable of specifically hybridizing to a target nucleic acid, i.e., the mRNA or the amplified DNA, can be labeled by any one of several methods typically used to detect the presence of hybridized nucleic acids. One common method of detection is the use of autoradiography using probes labeled with ³H, ¹²⁵I, ³⁵S, ¹⁴c, or ³²P, or the like. The choice of radioactive isotope depends on research preferences due to ease of synthesis, stability, and half lives of the selected isotopes. Other labels include compounds (e.g., biotin and digoxigenin), which bind to antiligands or antibodies labeled with fluorophores, chemiluminescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers necessary for practicing the present invention can be synthesized and labeled using well known techniques. Oligonucleotides used as probes and primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom., 255:137-149, 1983.

### IV. Quantitation of Polypeptides

### A. Obtaining Samples

The first step of practicing the present invention is to obtain a sample containing lymphoblasts from a subject being tested, assessed, or monitored for a blood cancer, the risk of developing the blood cancer, or the severity/progression/chances of recurrence of the condition as well as the likelihood of death due to the condition. Samples of the same type should be taken from both a control group (normal individuals not suffering from any blood disorder especially malignancy) and a test group (subjects being tested for a possible blood cancer, for example, ALL). Standard procedures routinely employed in hospitals or clinics are typically followed for this purpose, as stated in the previous section.

For the purpose of detecting the presence of a blood cancer or assessing the risk of developing a blood cancer in test subjects, individual patients' lymphoblast samples may be taken and the level of human CD9 protein may be measured and then compared to a standard control. If an increase in the level of human CD9 protein is observed when compared to the control level, the test subject is deemed to have a blood cancer such as leukemia or have an elevated risk of developing the disease. For the purpose of monitoring disease progression or assessing therapeutic effectiveness in blood cancer patients, individual patient's lymphoblast samples may be taken at different time points, such that the level of CD9 protein can be measured to provide information indicating the state of disease. For instance, when a patient's CD9 protein level shows a general trend of decrease over time, the patient is deemed to be improving in the severity of the blood cancer or the therapy the patient has been receiving is deemed effective. A lack of change in a patient's CD9 protein level or a continuing trend of increase on other hand would indicate a worsening of the condition and ineffectiveness of the therapy given to the patient. Generally, a higher CD9 protein level seen in a patient's lymphoblasts indicates a more severe form of the blood cancer the patient is suffering from and a worse prognosis of the disease, as manifested in shorter life expectancy, higher level of resistance to therapy, higher chances of recurrence, etc. Among blood cancer patients, for example those having received a diagnosis of ALL, one who has a higher level of CD9 protein expression in the lymphoblast sample than that found in a second ALL patient has a higher likelihood of disease recurrence compared to the second patient for any defined time period, such as 1, 2, 3, 4, 5, or up to 10 years post-diagnosis.

### B. Preparing Samples for CD9 Protein Detection

The peripheral blood or bone marrow sample from a subject is suitable for the present invention and can be obtained by well-known methods routinely employed by physicians and other medical professionals. In certain applications of this invention, peripheral blood samples may be the preferred sample type before lymphoblasts are cultured for further analysis.

### C. Determining the Level of CD9 Protein

A protein of any particular identity, such as CD9 protein, can be detected using a variety of immunological assays. In some embodiments, a sandwich assay can be performed by capturing the polypeptide from a test sample with an antibody having specific binding affinity for the polypeptide. The polypeptide then can be detected with a labeled antibody having specific binding affinity for it. Such immunological assays can be carried out using microfluidic devices such as microarray protein chips. A protein of interest (e.g., human CD9 protein) can also be detected by gel electrophoresis (such as 2-dimensional gel electrophoresis) and western blot analysis using specific antibodies. Alternatively, standard immunohistochemical techniques can be used to detect a given protein (e.g., human CD9 protein), using the appropriate antibodies. Both monoclonal and polyclonal antibodies (including antibody fragment with desired binding specificity) can be used for specific detection of the polypeptide. Such antibodies and their binding fragments with specific binding affinity to a particular protein (e.g., human CD9 protein) can be generated by known techniques. Because CD9 is a cell surface protein, its detection and quantification can also be achieved via high-throughput cell sorting techniques such as flow cytometry after the cells (lymphoblasts) have been incubated with an anti-CD9 antibody labeled with an appropriate fluorophore. Under such circumstances, the level of CD9 protein expression may also be indicated in the form of the number or percentage of CD9+lymphoblasts.

Other methods may also be employed for measuring the level of CD9 protein in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### V. Establishing a Standard Control

In order to establish a standard control for practicing the method of this invention, a group of healthy persons free of any blood disease (especially any form of blood cancers such as leukemia) as conventionally defined is first selected. These individuals are within the appropriate parameters, if applicable, for the purpose of screening for and/or monitoring blood cancers using the methods of the present invention. Optionally, the individuals are of same gender, similar age, or similar ethnic background.

The healthy status of the selected individuals is confirmed by well established, routinely employed methods including but not limited to general physical examination of the individuals and general review of their medical history.

Furthermore, the selected group of healthy individuals must be of a reasonable size, such that the average amount/concentration of CD9 mRNA or CD9 protein in the lymphoblasts obtained from the group can be reasonably regarded as representative of the normal or average level among the general population of healthy people. Preferably, the selected group comprises at least 10 human subjects.

Once an average value for the CD9 mRNA or protein is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### VI. Treatment of Blood Cancers

By illustrating the correlation of over-expression of CD9 mRNA/protein and blood cancers, especially leukemias such as ALL, the present invention further provides a means for treating patients suffering from a blood cancer: by way of suppressing CD9 mRNA or protein expression or inhibiting CD9 protein's biological activity. As used herein, treatment of a blood cancer encompasses reducing, reversing, lessening, or eliminating one or more of the symptoms of the blood cancer, as well as preventing or delaying the onset of one or more of the relevant symptoms, including reducing mortality or likelihood of disease recurrence among patients who have already received initial treatment. Inhibitors of CD9 can be of virtually any chemical and structural nature: they may be polypeptides (e.g., antibody, antibody fragment, aptamer), polynucleotides (e.g., antisense DNA/RNA, small inhibitory RNA, or micro RNA), and small molecules. As long as they possess confirmed inhibitory effect against CD9 expression or activity, such inhibitors may be useful for inhibiting blood cancer cell proliferation and therefore useful for treating blood cancers.

In addition, other means for treating blood malignancies, especially leukemias such as ALL, may be employed once a patient is diagnosed as suffering from a blood cancer by a method described herein. For instance, any one or more of the following may be used to treat an ALL patient: Abitrexate (Methotrexate), Arranon (Nelarabine), Asparaginase Erwinia chrysanthemi, Blinatumomab. Blincyto (Blinatumomab), Cerubidine (Daunorubicin Hydrochloride), Clafen (Cyclophosphamide), Clofarabine, Clofarex (Clofarabine), Clolar (Clofarabine), Cyclophosphamide, Cytarabine, Cytosar-U (Cytarabine), Cytoxan (Cyclophosphamide), Dasatinib, Daunorubicin Hydrochloride, Doxorubicin Hydrochloride, Erwinaze (Asparaginase Erwinia Chrysanthemi), Folex (Methotrexate), Folex PFS (Methotrexate), Gleevec (Imatinib Mesylate), Iclusig (Ponatinib Hydrochloride), Imatinib Mesylate, Marqibo (Vincristine Sulfate Liposome), Mercaptopurine, Methotrexate, Methotrexate LPF (Methorexate), Mexate (Methotrexate), Mexate-AQ (Methotrexate), Nelarabine, Neosar (Cyclophosphamide), Oncaspar (Pegaspargase), Pegaspargase, Ponatinib Hydrochloride, Prednisone, Purinethol (Mercaptopurine), Purixan (Mercaptopurine), Rubidomycin (Daunorubicin Hydrochloride), Sprycel (Dasatinib), Tarabine PFS (Cytarabine), Vincasar PFS (Vincristine Sulfate), and Vincristine Sulfate Liposome, or the combination treatment of Hyper-CVAD; and one or more of the following may be used to treat a patient diagnosed with acute myeloid leukemia (AML): Arsenic Trioxide, Cerubidine (Daunorubicin Hydrochloride), Clafen (Cyclophosphamide), Cyclophosphamide, Cytarabine, Cytosar-U (Cytarabine), Cytoxan (Cyclophosphamide), Daunorubicin Hydrochloride, Doxorubicin Hydrochloride, Idamycin (Idarubicin Hydrochloride), Idarubicin Hydrochloride, Midostaurin, Mitoxantrone Hydrochloride, Neosar (Cyclophosphamide), Rubidomycin (Daunorubicin Hydrochloride), Rydapt (Midostaurin), Tabloid (Thioguanine), Tarabine PFS (Cytarabine), Thioguanine, Trisenox (Arsenic Trioxide), Vincasar PFS (Vincristine Sulfate), and Vincristine Sulfate; and one or more of the following may be used to treat a patient diagnosed with chronic lymphocytic leukemia (CLL): Alemtuzumab, Ambochlorin (Chlorambucil), Amboclorin (Chlorambucil), Arzerra (Ofatumumab), Bendamustine Hydrochloride, Campath (Alemtuzumab), Chlorambucil, Clafen (Cyclophosphamide), Cyclophosphamide, Cytoxan (Cyclophosphamide), Fludara (Fludarabine Phosphate), Fludarabine Phosphate, Gazyva (Obinutuzumab), Ibrutinib, Idelalisib, Imbruvica (Ibrutinib), Leukeran (Chlorambucil), Linfolizin (Chlorambucil), Mechlorethamine Hydrochloride, Mustargen (Mechlorethamine Hydrochloride), Neosar (Cyclophosphamide), Obinutuzumab, Ofatumumab, Prednisone, Rituxan (Rituximab), Rituximab, Treanda (Bendamustine Hydrochloride), Venclexta (Venetoclax), Venetoclax, and Zydelig (Idelalisib); and one or more of the following may be used to treat a patient diagnosed with chronic myelogenous leukemia (CML): Bosulif (Bosutinib), Bosutinib, Busulfan, Busulfex (Busulfan), Clafen (Cyclophosphamide), Cyclophosphamide, Cytarabine, Cytosar-U (Cytarabine), Cytoxan (Cyclophosphamide), Dasatinib, Gleevec (Imatinib Mesylate), Hydrea (Hydroxyurea), Hydroxyurea, Iclusig (Ponatinib Hydrochloride), Imatinib Mesylate, Mechlorethamine Hydrochloride, Mustargen (Mechlorethamine Hydrochloride), Myleran (Busulfan), Neosar (Cyclophosphamide), Nilotinib, Omacetaxine Mepesuccinate, Ponatinib Hydrochloride, Sprycel (Dasatinib), Synribo (Omacetaxine Mepesuccinate), Tarabine PFS (Cytarabine), and Tasigna (Nilotinib).

### A. Suppressing CD9 Expression or Activity

1. Inhibitors of CD9 mRNA

Suppression of CD9 expression can be achieved through the use of nucleic acids siRNA, microRNA, miniRNa, lncRNA, antisense oligonucleotides, aptamer. Such nucleic acids can be single-stranded nucleic acids (such as mRNA) or double-stranded nucleic acids (such as DNA) that can translate into an active form of inhibitor of CD9 mRNA under appropriate conditions.

In one embodiment, the CD9 inhibitor-encoding nucleic acid is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in blood cells, especially in lymphoblasts. Administration of such nucleic acids can suppress CD9 expression in the target tissue, e.g., lymphoblasts. Since the human CD9 gene sequence encoding its mRNA is known as GenBank Accession No. NM_001769 and provided herein as SEQ ID NO:1, one can devise a suitable CD9-suppressing nucleic acid from the sequence, species homologs, and variants of these sequences.

### 2. Inhibitors of CD9 Protein

Suppression of CD9 protein activity can be achieved with an agent that is capable of inhibiting the activity of CD9 protein. Since CD9, a cell surface protein, is known to complex with other cell surface proteins, an in vitro assay can be used to screen for potential inhibitors of CD9 protein activity based in the binding between CD9 protein and a candidate compound. Once a compound is identified in the binding assay, further testing may be conducted to confirm and verify the compounds capability to inhibiting CD9 protein activity. In general, such an assay can be performed in the presence of CD9 protein or a fragment thereof, for example, a recombinantly produced CD9 protein or fragment, under conditions permitting its binding to a potential binding partner. For convenience, the CD9 protein or the candidate compound may be immobilized onto a solid support and/or labeled with a detectable moiety. A third molecule, such as an antibody (which may include a detectable label) to CD9 protein, can also be used to facilitate detection.

In some cases, the binding assays can be performed in a cell-free environment; whereas in other cases, the binding assays can be performed within a cell or on the cell surface, for example, using cells recombinantly or endogenously expressing an appropriate CD9 polypeptide.

The anti-cancer effects of a CD9 protein inhibitor of the present invention can also be demonstrated in in vivo assays. For example, a CD9 protein inhibitor can be injected into animals that have a compromised immune system (e.g., nude mice, SCID mice, or NOD/SCID mice) and therefore permit xenograft tumors. Injection methods can be subcutaneous, intramuscular, intravenous, intraperitoneal, or intratumoral in nature. Cancer development is subsequently monitored by various means, such as measuring cancer cell proliferation and disease relapse/recurrence, in comparison with a control group of animals with the same or similar disease but not given the inhibitor. The Examples section of this disclosure provides detailed description of some exemplary in vivo assays. An inhibitory effect is detected when a negative effect on cancerous blood cell proliferation or disease recurrence is established in the test group. Preferably, the negative effect is at least a 10% decrease; more preferably, the decrease is at least 20%, 30%, 40%, 50%, 60%, 70%, 80%, or 90%.

As stated above, CD9 protein inhibitors can have diverse chemical and structural features. For instance, an inhibitor can be a non-functional CD9 protein mutant that retaining the binding ability of CD9 protein to its cofactors or other binding partners, an antibody to the CD9 protein that interferes with CD9 protein activity (e.g., a neutralizing antibody such as ALB6), or any small molecule or macromolecule that simply hinders the interaction between CD9 protein and its cofactors or other binding partners. Essentially any chemical compound can be tested as a potential inhibitor of CD9 protein activity. Most preferred are generally compounds that can be dissolved in aqueous or organic (especially DMSO-based) solutions. Inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### B. Pharmaceutical Compositions

### 1. Formulations

Compounds of the present invention are useful in the manufacture of a pharmaceutical composition or a medicament. A pharmaceutical composition or medicament can be administered to a subject for the treatment of a blood cancer.

Compounds used in the present invention, e.g., an inhibitor of CD9 mRNA or protein (e.g., a neutralizing antibody against CD9 protein, such as ALB6), a nucleic acid encoding a polynucleotide or polypeptide inhibitor for CD9 gene expression or CD9 protein activity (e.g., an expression vector encoding a neutralizing antibody against CD9 protein), are useful in the manufacture of a pharmaceutical composition or a medicament comprising an effective amount thereof in conjunction or mixture with excipients or carriers suitable for application.

An exemplary pharmaceutical composition for suppressing CD9 expression comprises (i) an express cassette comprising a polynucleotide sequence encoding an inhibitor of CD9 protein as described herein, and (ii) a pharmaceutically acceptable excipient or carrier. The terms pharmaceutically-acceptable and physiologically-acceptable are used synonymously herein. The expression cassette may be provided in a therapeutically effective dose for use in a method for treatment as described herein.

A CD9 inhibitor or a nucleic acid encoding a CD9 inhibitor can be administered via liposomes, which serve to target the conjugates to a particular tissue, as well as increase the half-life of the composition. Liposomes include emulsions, foams, micelles, insoluble monolayers, liquid crystals, phospholipid dispersions, lamellar layers and the like. In these preparations the inhibitor to be delivered is incorporated as part of a liposome, alone or in conjunction with a molecule which binds to, e.g., a receptor prevalent among the targeted cells (e.g., lymphoblasts), or with other therapeutic or immunogenic compositions. Thus, liposomes filled with a desired inhibitor of the invention can be directed to the site of treatment, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, parenterally, or rectally.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

### 2. Routes of Administration

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Suitable formulations for transdermal application include an effective amount of a compound or agent of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a CD9 inhibitor or a nucleic acid encoding a CD9 inhibitor, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds and agents of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the active ingredient, e.g., a CD9 inhibitor or a nucleic acid encoding a CD9 inhibitor, may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The inhibitors can also be formulated in rectal compositions, for example, suppositories or retention enemas, for example, containing conventional suppository bases, for example, cocoa butter or other glycerides.

Furthermore, the active ingredient can be formulated as a depot preparation. Such long-acting formulations can be administered by implantation (for example, subcutaneously or intramuscularly) or by intramuscular injection. Thus, for example, the active ingredient can be formulated with suitable polymeric or hydrophobic materials (for example as an emulsion in an acceptable oil) or ion exchange resins, or as sparingly soluble derivatives, for example, as a sparingly soluble salt.

A pharmaceutical composition or medicament of the present invention comprises (i) an effective amount of a compound as described herein that decreases the level or activity of CD9 protein, and (ii) another therapeutic agent. When used with a compound of the present invention, such therapeutic agent may be used individually, sequentially, or in combination with one or more other such therapeutic agents (e.g., a first therapeutic agent, a second therapeutic agent, and a compound of the present invention). Administration may be by the same or different route of administration or together in the same pharmaceutical formulation.

### 3. Dosage

Pharmaceutical compositions or medicaments can be administered to a subject at a therapeutically effective dose to prevent, treat, or control a blood cancer as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular compound in a particular subject. For example, each type of CD9 inhibitor or nucleic acid encoding a CD9 inhibitor will likely have a unique dosage. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

To achieve the desired therapeutic effect, compounds or agents may be administered for multiple days at the therapeutically effective daily dose. Thus, therapeutically effective administration of compounds to treat a pertinent condition or disease described herein in a subject requires periodic (e.g., daily) administration that continues for a period ranging from three days to two weeks or longer. Typically, agents will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

Optimum dosages, toxicity, and therapeutic efficacy of such compounds or agents may vary depending on the relative potency of individual compounds or agents and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any agents used in the methods of the invention, the therapeutically effective dose can be estimated initially from cell culture assays. A dose can be formulated in animal models to achieve a circulating plasma concentration range that includes the IC₅₀ (the concentration of the agent that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of agents is from about 1 ng/kg to 100 mg/kg for a typical subject.

Exemplary dosages for a CD9 inhibitor or a nucleic acid encoding a CD9 inhibitor described herein are provided. Dosage for a CD9 inhibitor-encoding nucleic acid, such as an expression cassette, can be between 0.1-0.5 mg/eye, with intravitreous administration (e.g., 5-30 mg/kg). Small organic compounds inhibitors can be administered orally at between 5-1000 mg, or by intravenous infusion at between 10-500 mg/ml. Monoclonal antibody inhibitors can be administered by intravenous injection or infusion at 50-500 mg/ml (over 120 minutes); 1-500 mg/kg (over 60 minutes); or 1-100 mg/kg (bolus) five times weekly. CD9 protein or mRNA inhibitors can be administered subcutaneously at 10-500 mg; 0.1-500 mg/kg intravenously twice daily, or about 50 mg once weekly, or 25 mg twice weekly.

Pharmaceutical compositions of the present invention can be administered alone or in combination with at least one additional therapeutic compound. Exemplary advantageous therapeutic compounds include systemic and topical anti-inflammatories, pain relievers, anti-histamines, anesthetic compounds, and the like. The additional therapeutic compound can be administered at the same time as, or even in the same composition with, main active ingredient (e.g., a CD9 inhibitor or a nucleic acid encoding a CD9 inhibitor). The additional therapeutic compound can also be administered separately, in a separate composition, or a different dosage form from the main active ingredient. Some doses of the main ingredient, such as a CD9 inhibitor or a nucleic acid encoding a CD9 inhibitor, can be administered at the same time as the additional therapeutic compound, while others are administered separately, depending on the particular symptoms and characteristics of the individual.

The dosage of a pharmaceutical composition of the invention can be adjusted throughout treatment, depending on severity of symptoms, frequency of recurrence, and physiological response to the therapeutic regimen. Those of skill in the art commonly engage in such adjustments in therapeutic regimen.

### VII. Kits and Devices

The invention provides compositions and kits for practicing the methods described herein to assess CD9 level, at the level of both CD9 mRNA and protein in lymphoblasts obtained from a subject, which can be used for various purposes such as detecting or diagnosing the presence of a blood cancer (especially leukemia such as ALL), determining the risk of developing a blood cancer, and monitoring the progression of the blood cancer in a patient, including assessing the likelihood of recurrence of the blood cancer among patients who have received a diagnosis of the disease and may have been treated, e.g., by surgery, chemotherapy, and/or radiotherapy.

Kits for carrying out assays for determining CD9 mRNA level typically include at least one oligonucleotide useful for specific hybridization with at least one segment of the CD9 coding sequence or its complementary sequence. Optionally, this oligonucleotide is labeled with a detectable moiety. In some cases, the kits may include at least two oligonucleotide primers that can be used in the amplification of at least one segment of CD9 mRNA (e.g., SEQ ID NO:1) by PCR, particularly by RT-PCR.

Kits for carrying out assays for determining CD9 protein level typically include at least one antibody useful for specific binding to the CD9 protein amino acid sequence. Optionally, this antibody is labeled with a detectable moiety. The antibody can be either a monoclonal antibody or a polyclonal antibody. Some exemplary antibodies against CD9 include clones ALB6 (Beckman Coulter), M-L13 (BD Biosciences), and MM2/57 (Millipore), with ALB6 being a particularly effective neutralizing antibody. In some cases, the kits may include at least two different antibodies, one for specific binding to the CD9 protein (i.e., the primary antibody) and the other for detection of the primary antibody (i.e., the secondary antibody), which is often attached to a detectable moiety.

Typically, the kits also include an appropriate standard control. The standard controls indicate the average value of CD9 protein or mRNA in lymphoblasts of healthy subjects not suffering from any blood cancer or any increased risk of developing a blood cancer. In some cases such standard control may be provided in the form of a set value. In addition, the kits of this invention may provide instruction manuals to guide users in analyzing test samples and assessing the presence, risk, or likelihood of recurrence of blood cancers in a test subject.

In a further aspect, the present invention can also be embodied in a device or a system comprising one or more such devices, which is capable of carrying out all or some of the method steps described herein. For instance, in some cases, the device or system performs the following steps upon receiving a lymphoblast sample, e.g., a processed bone marrow or peripheral blood sample taken from a subject being tested for detecting a blood cancer, assessing the risk of developing a blood cancer, or monitored for progression of the condition: (a) determining in sample the amount or concentration of CD9 mRNA or protein; (b) comparing the mRNA or protein amount/concentration with a standard control value; and (c) providing an output indicating whether a blood cancer is present in the subject or whether the subject is at risk of developing a blood cancer, or whether there is a change, i.e., worsening or improvement, in the subject's blood cancer condition, or whether the patient has an increased likelihood of recurrence the blood cancer, e.g., after the initial diagnosis and/or treatment, or whether the patient has an increased likelihood of dying from the blood cancer within the next defined time period (e.g., the next 5 years). In other cases, the device or system of the invention performs the task of steps (b) and (c), after step (a) has been performed and the amount or concentration from (a) has been entered into the device. Preferably, the device or system is partially or fully automated.

### EXAMPLES

The following examples are provided by way of illustration only and not by way of limitation. Those of skill in the art will readily recognize a variety of non-critical parameters that could be changed or modified to yield essentially the same or similar results.

### Introduction

B-precursor acute lymphoblastic leukemia (B-ALL) is the most common childhood malignancy, accounting for approximately 30% of pediatric cancers.¹ It is presented as aberrant proliferation of B-precursor cells at different stages of maturation in the bone marrow (BM), with majority of cases exhibiting aneuploidy, harboring structural chromosomal rearrangements or submicroscopic genetic alterations that are important initiating events in leukemogenesis.² With advances in risk-adapted chemotherapy and supportive care, the overall cure rate of newly diagnosed B-ALL is approaching 85% in most developed countries.³ However, specific subtypes of B-ALL, including BCR-ABL1 and MLL-AF4 translocations, hypodiploidy, CRLF2 rearrangement, IKZF1 deletion and those with a BCR-ABL1-like gene expression profile have been associated with higher risk of treatment failure and poorer outcomes.⁴ Moreover, 15-20% of patients would experience relapse, and the prognosis of these patients remain inferior, with long-term survival rate of <50%.⁵ Further intensification of existing chemotherapeutic regimens is unlikely to improve the cure rate and may significantly increase toxicity.⁶ Long-term survivors of pediatric ALL are also at risk of therapy-related late effects.⁷ Thus, it is necessary to develop new targeted agents that can be incorporated into established chemotherapeutic regimens for improving the outcome of high-risk and relapsed/refractory B-ALL.

CD9, a tetraspanin family protein, regulates various physiologic processes such as cell motility and adhesion.⁸ It exerts its functions by associating with different transmembrane proteins to form tetraspanin-enriched microdomains, where the activities of specific binding partners are modulated.⁹ It was previously reported the genome-wide expression profile of cord blood hematopoietic stem/progenitor cells (HSPC), and identified CD9 as a downstream effector of the stromal cell-derived factor-1 (SDF-1)/C-X-C motif chemokine receptor 4 (CXCR4) axis. In addition, its role in regulating HSPC migration, adhesion and homing has been demonstrated.¹⁰ There has also been emerging evidence associating CD9 expression with metastasis and progression of various cancers.¹¹ For B-ALL, CD9⁺ cells were demonstrated to possess self-renewal properties and were enriched with leukemia-initiating cells.^{12,13} Moreover, knockdown of CD9 inhibited engraftment of B-ALL cell lines and prolonged the survival of transplanted animals.^{14,15} Yet, the impact of CD9 expression on prognosis of B-ALL patients, and the potential beneficial effect of applying CD9 antagonists for treatment of B-ALL remain unexplored. The objectives of our study were to: (i) characterize the expression of CD9 in a cohort of pediatric B-ALL patients and its association with clinical outcomes; (ii) evaluate the efficacy of using a CD9 neutralizing antibody for treatment of B-ALL in the NOD/SCID mouse xenograft model; and (iii) investigate the role of CD9 in regulation of leukemia-stroma interaction, focusing on the potential benefit of CD9 blockade for enhancing chemosensitivity.

### Materials and Methods

### BALL Patient Samples, BM Stromal Cells and Cell Lines

All human specimens were obtained with informed written consents and in accordance with procedures approved by the Joint CUHK-NTEC Clinical Research Ethics Committee of The Chinese University of Hong Kong. BM samples were collected from children with B-ALL during routine aspiration procedures for diagnostic purpose. Patients were stratified into standard-risk, intermediate-risk and high-risk groups according to presenting features described in our previously published studies.¹⁶ Primary stromal cells from healthy donors were recovered from blood filter sets of the BM collection kit (BioAccess), and cultured in MEM-α medium (Life Technologies) supplemented with 10% fetal bovine serum (FBS; Life Technologies). Mesenchymal stromal cells (CD45⁻CD90⁺CD105⁺CD166⁺) derived from the 3′ to 5^{th} passage were used for co-culture experiments. B-ALL cell lines SUP-B15 (ATCC), Reh (ATCC), 697 (DSMZ) and RS4;11 (DSMZ) were maintained in RPMI-1640 medium (Life Technologies) supplemented with 10% FBS.

### Characterization of CD9 Expression

Mononuclear cells were enriched from patient BM samples using Ficoll-Paque Plus (GE Healthcare). The expression of cell surface CD9 on leukemic blasts was characterized by staining with fluorochrome-conjugated antibodies against CD9 (M-L13), CD19 (HIB19), CD34 (8G12) and CD45 (J.33, Beckman Coulter), and assessed by flow cytometry (LSRFortessa, BD Biosciences). All antibodies were purchased from BD Biosciences unless otherwise specified. Data were analyzed by the FACS Diva (BD Biosciences) or FlowJo softwares (TreeStar).

### Xenograft Experiments

All animal experiments were conducted in accordance with procedures approved by the Animal Experimentation Ethics Committee of The Chinese University of Hong Kong. Immunodeficient NOD.CB17-Prkdc^{scid}/J (NOD/SCID) mice were used as a xenograft model of B-ALL.¹⁷ They were purchased from the Jackson Laboratory and bred in the Laboratory Animal Services Centre of The Chinese University of Hong Kong. To study the role of CD9 in leukemic cell homing and engraftment, 8- to 11-week-old female mice were sublethally irradiated (250 cGy, Gammacell 1000 Elite Irradiator, MDS Nordion) and intravenously infused with 697 cells (5×10⁶) 24 hours after irradiation. Prior to transplantation, 697 cells were incubated with CD9 antibodies [ALB6 (Beckman Coulter), M-L13 (BD Biosciences) or MM2/57 (Millipore); 10 μg/mL] or control IgG antibody (R&D Systems) for 4 hours at 37° C. In some experiments, mice were intraperitoneally injected with 200 μg of CD122 antibody (TM-β1, eBioscience) to deplete residual NK cells.¹⁸ Antibody-treated cells were monitored for apoptosis by Annexin V/7-AAD staining (BD Biosciences), phagocytic signals by detection of calreticulin (FMC75, Abcam) and CD47 (B6H12, BD Biosciences), and expression of homing receptors by antibodies against CXCR4 (12G5), integrins α4 (9F10) and β1 (MAR4) (BD Biosciences). Single-cell suspensions were then prepared from mouse BM, spleen, blood and liver at 20 hours (for analysis of homing) or at the indicated time points (for analysis of engraftment) after cell infusion. Fc receptors were blocked by anti-mouse CD16/CD32 (2.4G2, BD Biosciences) and human serum (Sigma-Aldrich). Leukemic cells were detected by flow cytometry using human-specific antibodies CD9, CD10 (HI10a), CD19 and CD45 (BD Biosciences). Dead cells were identified by 7-AAD staining and were excluded from analysis.

To study the effects of CD9 antibody on leukemic progression, NOD/SCID mice transplanted with B-ALL cell lines (5×10⁶) or patient B-ALL blasts (3.1×10⁶ to 5×10⁶) were randomized to receive intraperitoneal injection of CD9 antibody (ALB6; 20 μg/mouse) or control IgG antibody twice weekly for 2 weeks, starting on day 3 after B-ALL cell infusion.^{19,20} In chemotherapy experiments, 697-xenografted mice were randomized into 4 treatment groups: (i) IgG antibody alone, (ii) CD9 antibody alone, (iii) IgG+vincristine (0.8 mg/kg, Sigma Aldrich) and (iv) CD9 antibody+vincristine. To analyze animal survival, mice with ≧15% weight loss, obvious distress and impaired mobility were sacrificed and regarded as death.²¹ Leukemic cell engraftment in these moribund animals was confirmed by flow cytometry.

### Migration, Stromal Adhesion and VCAM-1 Binding Assays

Chemotaxis experiments were performed using Transwells (5 μm pore, Corning). B-ALL cell lines (2×10⁵) were seeded in the upper chamber, and medium containing 100 ng/mL SDF-1 (R&D Systems) was added to the lower chamber. After 4 hours at 37° C., migrated cells were counted with a hematocytometer. Adhesion of B-ALL cells to BM-derived stromal cells was performed in high-binding 96-well plates (Corning). B-ALL cell lines (2×10⁴ cells/well) were allowed to adhere to monolayers of stromal cells for 2 hours at 37° C. Non-adherent cells were enumerated. Integrin α4β1 activity was assessed by the ability of B-ALL cells to bind soluble vascular cell adhesion protein-1 (VCAM-1). Briefly, cells were incubated with VCAM-1-Fc (10 μg/mL, R&D Systems), followed by staining with goat-anti-human IgG-FITC or -APC (1:200, Jackson ImmunoResearch). The number of VCAM-1-bound cells was assessed by flow cytometry. In CD9 knockout experiments, 697 cells were transduced with lentivirus bearing the CD9-targeting single guide RNA (sgRNA, sequence: GGGATATTCCCACAAGGATG) and the Cas9 endonuclease, sorted for green fluorescent protein (GFP⁺) cells by FACSAria Fusion (BD Biosciences) and tested for VCAM-1 binding. A non-targeting sgRNA (sequence: GCACTCACATCGCTACATCA) served as the control.

### Co-Immunoprecipitation Assay

B-ALL cell lines were lysed in 1% Brij97 (Sigma Aldrich). Cell lysates (1.5 mg) were immunoprecipitated with 10 μg CD9 antibody (MM2/57) or control IgG antibody (R&D Systems) overnight at 4° C. Immune complexes were captured with protein A/G agarose (Pierce), washed, eluted and subjected to separation by SDS-PAGE. Immunoblots were probed with antibodies against CD9 (D801A, 1:3000), integrin α4 (D2E1, 1:1000) and integrin β1 (D2E5, 1:1000). The reactions were developed with peroxidase-conjugated secondary antibodies (1:5000) and the SignalFire Plus ECL Kit. All Western blot reagents were from Bio-Rad and antibodies were from Cell Signaling Technology.

### Apoptosis Assay

B-ALL cell lines (1×10⁵) were cultured with or without monolayers of BM stromal cells, and exposed to 10 μg/mL of control IgG or CD9 antibody (ALB6). Cells were then treated with vincristine (12.5 nM; Sigma Aldrich) for 3 days at 37° C. Apoptotic cell death was monitored using the Annexin V/7-AAD Apoptosis Detection Kit (BD Biosciences).

### Statistics

Demographic data were analyzed by Fisher's exact test or Mann-Whitney U test where appropriate. The Kaplan-Meier method was used to estimate OS (period from diagnosis to death from any cause) and RFS (period from diagnosis to first relapse or death from any cause). Comparison of survival outcomes between CD9⁺ and CD9⁻ patients were performed by log-rank test. The Cox proportional hazards model with backward stepwise regression was used to estimate the hazard ratio and significance for each prognostic factor in univariate and multivariate analyses.^{22,23} Comparisons of leukemic burden and animal survival in in vivo experiments were performed by unpaired Student's t-test and log-rank test, respectively. In vitro effects of CD9 antibody on migration, adhesion, integrin activity and apoptosis were analyzed by paired Student's t-test. P values of ≦0.05 were considered significant. Statistical analyses were performed using SPSS version 21.0.

### Results

### Expression of CD9 is Associated With Inferior Survival Outcomes in Pediatric B-ALL Patients

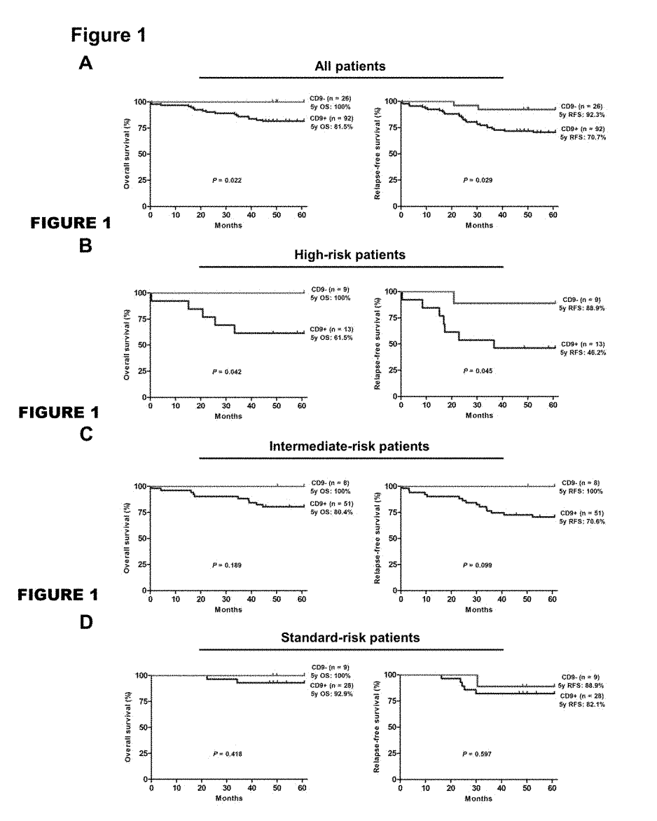

The expression pattern of cell surface CD9 on leukemic blasts of B-ALL patients was first examined by flow cytometry. Among 118 cases tested, blasts of 92 patients (78.0%) were CD9⁺ (≧20% of CD9-expressing blasts; Table 1). There were no significant differences in age, sex and white cell count between CD9⁺ and CD9⁻ patients. Major cytogenetics subgroups were similarly distributed except for hyperdiploidy (all patients were CD9⁺; P=0.022) and ETV6-RUNX1 translocation (higher prevalence in CD9⁻ patients; P=0.001). Significantly more CD9⁺ patients were stratified into the intermediate-risk group (P=0.044) and a higher proportion of CD9⁻ patients was stratified into the high-risk group (P=0.025). Besides, CD9⁻ patients had poorer prednisone response (P=0.014). Kaplan-Meier survival analysis was then performed to investigate the possible association of CD9 expression with clinical outcomes in pediatric B-ALL patients. The 5-year overall survival (OS) and relapse-free survival (RFS) rates of CD9⁺ patients were significantly lower than those in CD9⁻ patients (P≦0.029; FIG. 1A). Subgroup analysis revealed remarkably poorer outcomes in CD9⁺ patients of the high-risk group (P≦0.045; FIG. 1B). A similar trend was also observed in patients of the intermediate-risk group (FIG. 1C) but not in the standard-risk group (FIG. 1D). The potential roles of CD9 expression, age, risk group, white cell count and prednisone response were next evaluated as predictors of RFS in univariate and multivariate Cox regression models (Table 2). In univariate analysis, CD9 positivity, age <1 year, white cell count ≧100×10⁹/L and poor prednisone response were associated with lower RFS rate (P≦0.050). In multivariate analysis, CD9 positivity (HR=6.0; P=0.019) and poor prednisone response (HR=3.9; P=0.015) remained as independent prognostic factors for lower RFS rate.

### CD9 is Involved in B-ALL Homing and Engraftment

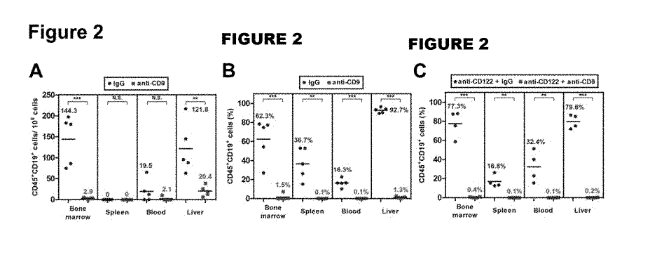

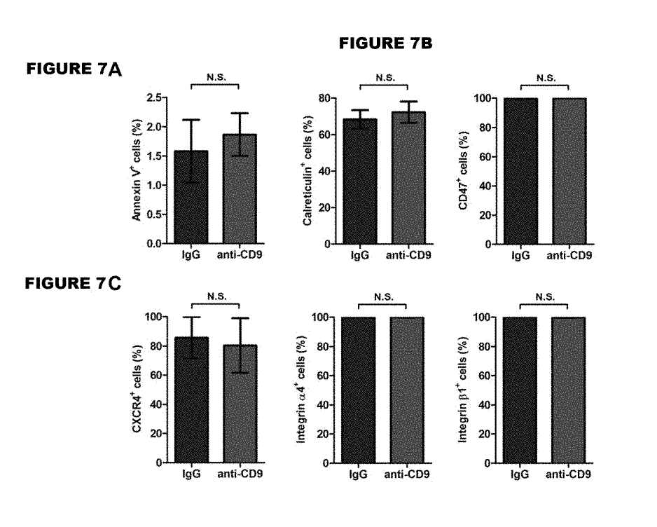

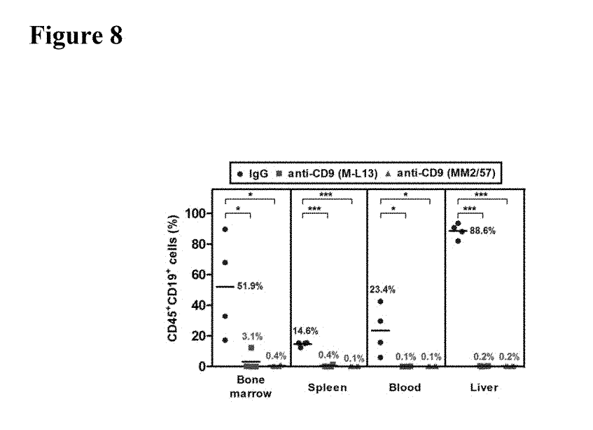

To address if CD9 could act on leukemic cell homing and engraftment, the B-ALL cell line 697 was treated with a neutralizing CD9 antibody (clone ALB6)^{10,24} or control IgG antibody before transplantation into NOD/SCID mice. The results showed that pretreatment with CD9 antibody significantly reduced homing of 697 cells to recipient BM and liver by 98.0% (P<0.001) and 83.2% (P=0.007), respectively, as indicated by the prevalence of human CD45⁺CD19⁺ cells at 20 hours post-transplantation (FIG. 2A). In vitro observations revealed that CD9 antibody did not directly induce apoptosis (FIG. 7A), alter pro-phagocytic (calreticulin) or anti-phagocytic signals (CD47) (FIG. 7B), and change the expression of major homing molecules including CXCR4, integrins α4 and β1 (FIG. 7C). It has also been shown that CD9 antibody pretreatment substantially reduced engraftment of 697 cells in BM, spleen, blood and liver of NOD/SCID mice by ≧97.6% at 16-20 days post-transplantation (P≦0.001; FIG. 2B). Similar inhibitory effects on 697 cell engraftment were observed using other clones (M-L13 and MM2/57) of CD9 antibodies (FIG. 8). In mice depleted of NK cells by CD122 antibody, CD9 blockade exerted a comparable effect on engraftment of 697 cells (FIG. 2C). These data ruled out the possibility that the CD9 antibody-treated cells were eliminated by NK cells via antibody-dependent cellular cytotoxicity (ADCC).

### Administration of CD9 Antibody Reduces Leukemic Burden and Prolongs Survival of NOD/SCID Mice Xenografted With B-ALL Cell Lines

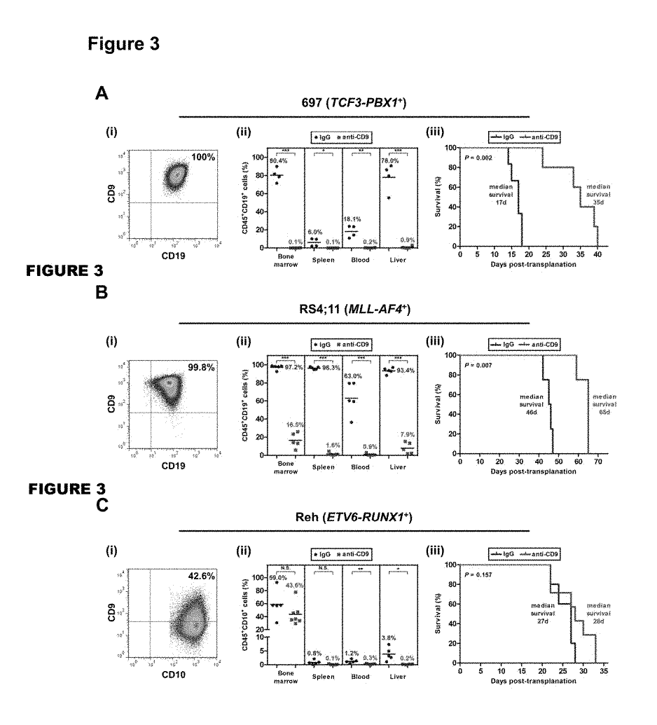

Given the potent activity of CD9 antibody in suppressing leukemic cell homing and engraftment, a more clinically relevant approach we then explored by administering CD9 antibody at 3, 7, 10 and 14 days after leukemic cell transplantation, using 3 B-ALL cell lines with different cytogenetic abnormalities. These results showed that in vivo treatment with CD9 antibody substantially reduced leukemic burden of the CD9^{high}, TCF3-PBX1⁺697 cells in all tested organs by ≧98.9% (P≦0.037; FIG. 3, Ai and Aii). This treatment protocol also significantly prolonged survival of 697-xenografted mice from 17 days to 35 days (P=0.002; FIG. 3Aiii). In the CD9^{high}, MLL-AF4⁺RS4;11 xenograft model, administration of CD9 antibody significantly reduced leukemic burden by ≧82.9% (P<0.001; FIG. 3, Bi and Bii) and prolonged survival of transplanted mice from 46 days to 65 days (P=0.007; FIG. 3Biii). In the CD9^{low}, ETV6-RUNX1⁺ Reh xenograft, CD9 antibody treatment only moderately decreased leukemic burden in blood and liver of transplanted mice (P≦0.019) but not in BM and spleen (FIG. 3, Ci and Cii). Moreover, administration of CD9 antibody conferred no significant improvement in survival duration of the Reh-transplanted animals (FIG. 3Ciii).

### CD9 Antibody is Effective for Treatment of Mice Xenografted With Patient-Derived B-ALL Cells

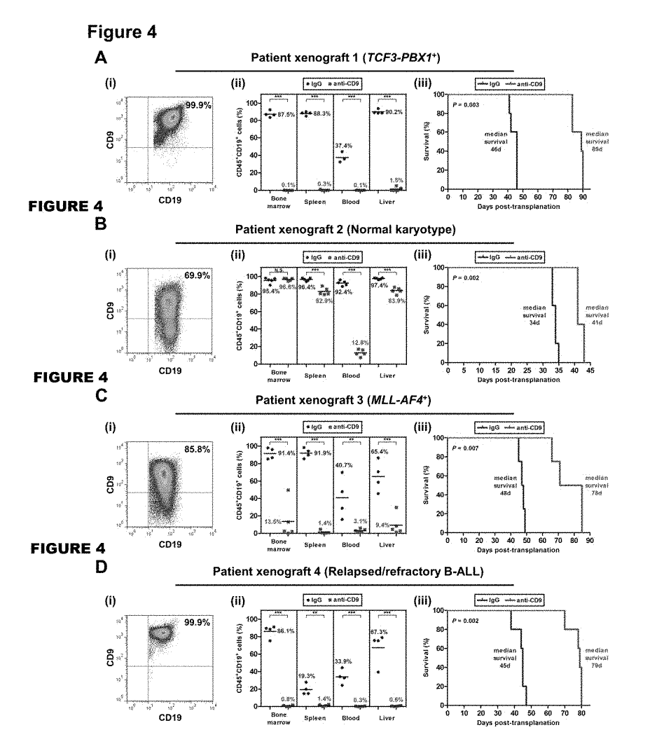

The efficacy of CD9 antibody for treatment of primary B-ALL blast-xenografted mice was evaluated by using 3 patient BM samples collected at diagnosis (FIG. 4, A-C) and a relapsed/refractory sample collected from a patient who failed 2 consecutive courses of chemotherapy (FIG. 4D). The clinical characteristics of these patients are shown in Table 3. All of the transplanted samples expressed cell surface CD9 (FIG. 4, Ai-Di). Treatment with CD9 antibody substantially reduced leukemic load in mice transplanted with the intermediate-risk, TCF3-PBX1⁺ B-ALL cells by ≧98.3% (P<0.001; FIG. 4Aii) and prolonged animal survival from 46 days to 89 days (P=0.003; FIG. 4Aiii). However, CD9 antibody only modestly decreased leukemic burden in the spleen, blood and liver (P<0.001), but not in the BM of mice xenografted with the intermediate-risk B-ALL cells with normal karyotype (FIG. 4Bii). Survival durations of this group of CD9 antibody-treated mice were mildly prolonged from 34 days to 41 days (P=0.002; FIG. 4Biii). Importantly, CD9 antibody treatment was also effective for high-risk B-ALL subtypes. Specifically, administration of CD9 antibody significantly decreased leukemic burden by ≧82.2% (P≦0.009; FIG. 4Cii) and prolonged survival of mice transplanted with the high-risk infantile MLL-AF4⁺ B-ALL from 48 days to 78 days (P=0.007; FIG. 4Ciii). Similar efficacy was demonstrated in the relapsed/refractory case, with leukemic burden being decreased by ≧93.1% (P≦0.001; FIG. 4Dii) and animal survival being significantly prolonged from 45 days to 79 days (P=0.002; FIG. 4Diii).

### CD9 Antibody Enhances Chemosensitivity of BALL Cells in Stromal Co-Culture and Xenograft Models

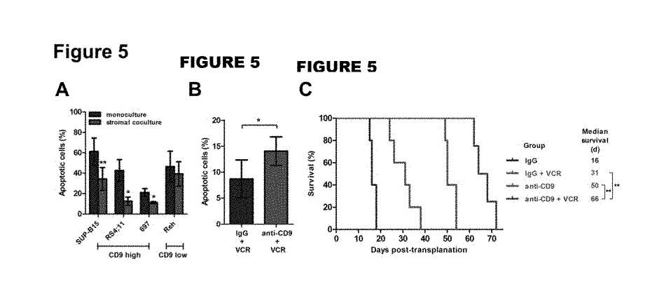

To explore the association of CD9 expression with chemosensitivity, the response of B-ALL cell lines to vincristine was compared in the presence or absence of primary BM stromal cells. The results showed that stromal co-culture reduced vincristine-mediated apoptotic cell death in CD9^{high} cell lines SUP-B15 (P=0.006), RS4;11 (P=0.029) and 697 (P=0.041), whereas the protective effect of stromal cells was absent in the CD9^{low} Reh cells (FIG. 5A). Addition of CD9 antibody significantly enhanced vincristine-induced apoptosis of 697 cells in stromal co-cultures (P=0.016; FIG. 5B). Importantly, combined treatment of 697-xenografted mice with CD9 antibody and vincristine significantly prolonged animal survival to 66 days, comparing with those treated with IgG+vincristine (31 days; P=0.005) or CD9 antibody alone (50 days; P=0.006; FIG. 5C).

### CD9 Regulates Migration, Adhesion and Integrin Affinity of B-ALL Cells

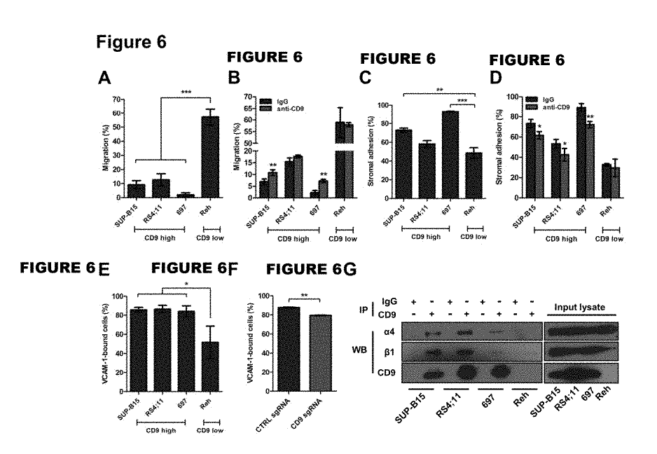

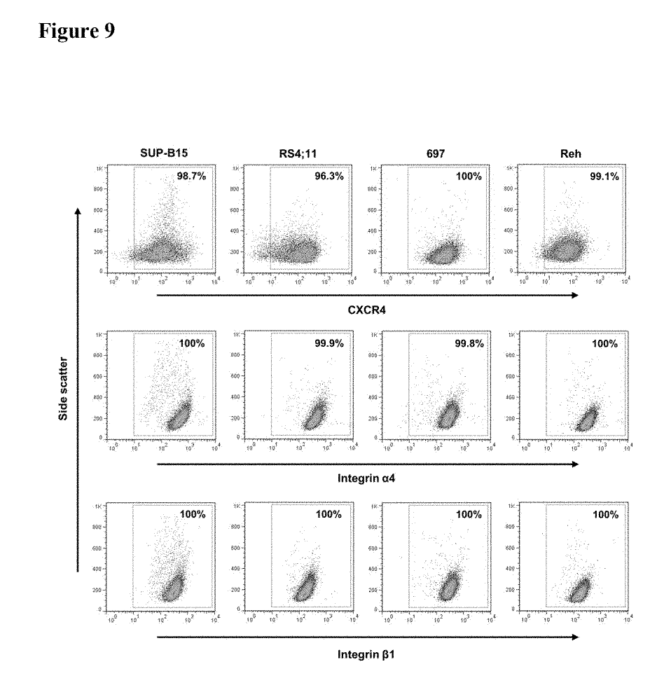

CD9 has been implicated in the regulation of cell motility and adhesion.^{25,26} The effect of CD9 on transwell migration and stromal adhesion activity of B-ALL cell lines was therefore investigated. The results showed that CD9^{high} SUP-B15, RS4;11 and 697 cells migrated less efficiently than CD9^{low} Reh cells to a SDF-1 gradient (P<0.001; FIG. 6A). This phenomenon was independent of CXCR4 expression (FIG. 9). In CD9^{high} cell lines SUP-B15 and 697, migration activities could be modestly enhanced by pretreatment with the CD9 neutralizing antibody (P≦0.008; FIG. 6B). In addition, stronger adhesion activities on BM-derived stromal cells were found in CD9^{high} B-ALL cell lines SUP-B15 and 697 (P≦0.002; FIG. 6C), which were inhibited by CD9 antibody (P≦0.019; FIG. 6D). It has been reported that the adhesive functions of CD9 was mediated by binding and modulating the activity of integrins.^{27,28} The affinity of integrin very late antigen-4 (VLA-4, composed of α4β1 subunits) among B-ALL cell lines was thus compared with different CD9 expression levels using a VCAM-1 binding assay. Although all 4 cell lines expressed similar levels of integrins α4 and β1 (FIG. 9), the CD9^{high} SUP-B15, RS4;11 and 697 cells preferentially bound to soluble VCAM-1 (≧84.2% binding), when compared to the CD9^{low} cell line Reh (51.6% binding; P≦0.011; FIG. 6E). Using the CRISPR/Cas9 system, we also showed that CD9 knockout significantly inhibited VCAM-1 binding in 697 cells (P=0.003; FIG. 6F). Co-immunoprecipitation experiments revealed that CD9 physically interacted with α4 and β1 integrins in the CD9^{high} cell lines SUP-B15, RS4;11 and 697 (FIG. 6G).

### Discussion

This study provided the first evidence that targeting CD9 with a neutralizing antibody effectively reduced B-ALL burden and significantly prolonged animal survival in the preclinical NOD/SCID mouse xenograft model. Importantly, co-administration of CD9 antibody enhanced the treatment efficacy of chemotherapy in these animals. In addition, it has been demonstrated that expression of CD9 was associated with inferior outcomes in pediatric B-ALL patients. Mechanistic investigations revealed that CD9 associated physically with integrin VLA-4 in B-ALL cells, and regulated their interaction with BM stroma. These findings indicate that CD9 therapeutic antibodies can be developed as a novel and promising agent for treatment of high-risk and relapsed/refractory B-ALL.

In a cohort of 118 patients with clinical characteristics comparable to international studies,^{29,30} it was revealed that CD9 positivity was significantly associated with inferior clinical outcomes, and the phenomenon was especially prominent in patients of the high-risk group. These data suggest that CD9 expression could be used in conjunction with other known prognostic factors for refinement of risk group stratification. Although the prognostic impact of CD9 has not been previously reported in pediatric or adult leukemia, CD9 expression was associated with disease progression in other malignancies. In breast,³¹ gastric³² and lung cancers,³³ expression of CD9 conferred increased risks of metastasis and chemoresistance. In contrast, the impact of CD9 on some cancer types appeared to occur in the opposite direction. In cervical carcinoma,³⁴ prostate cancer,³⁵ pancreatic cancer³⁶ and multiple myeloma,³⁷ the loss of CD9 expression was associated with metastasis and tumor progression. Indeed, the involvement of CD9 in malignant progression remains unclear and is likely governed by intrinsic mechanisms of specific cancer cell types and association of CD9 with different partner proteins.³⁸ For examples, the association of CD9 with EWI-2 could inhibit metastasis of melanoma,³⁹ whereas the association of CD9 with MMP-9 promoted the invasive phenotype of fibrosarcoma.⁴⁰

Using the NOD/SCID mouse model, the inventors have demonstrated potent activities of CD9 antibody against a wide spectrum of B-ALL harboring specific chromosomal translocations and leukemia risks. Importantly, high- or intermediate-risk subtypes including relapsed/refractory B-ALL and those harboring TCF3-PBX1 and MLL-AF4 translocations were sensitive to CD9 antibody. In contrast, treatment outcome was modest in normal karyotype B-ALL and essentially insignificant in the standard-risk, ETV6-RUNX1⁺ cell line Reh, which expressed a moderate level of CD9. It remains to be determined whether the extent of antibody response is dependent on CD9 expression level or the intrinsic properties of B-ALL subtypes. These results are in line with two independent groups who reported that shRNA knockdown of CD9 inhibited engraftment of B-ALL cell lines NALM-6 and YAWN90 in xenografted animals.^{14,15} Using the stromal co-culture and xenograft models, we provided additional evidence that CD9 antibody could enhance sensitivity of B-ALL cells to vincristine. Chemotherapy refractoriness remains a clinical challenge in relapsed patients and those with high-risk features such as MLL-rearrangements.^{41,42} The administration of CD9 antibody thus represented a novel and clinically feasible approach and may potentially be developed as an adjunct to conventional chemotherapy for treatment of B-ALL. The intervention will be particularly beneficial for CD9⁺ patients with high-risk or relapsed/refractory disease.

In preclinical animal models, the CD9 neutralizing antibody ALB6 has been shown to inhibit the growth of ovarian,⁴³ gastric⁴⁴ and colon cancers.⁴⁵ However, there is limited knowledge regarding its mechanisms of action. Hwang et al⁴³ and Murayama et al⁴⁶ showed that ALB6 could trigger apoptosis through modulation of NF-KB, JNK and p38 pathways. In contrast, the in vitro data showed that the effects of ALB6 were not mediated by direct induction of apoptosis. In addition, this study provided evidence that CD9 antibody-treated cells were not eliminated through ADCC or modulation of phagocytic signals, which were reported mechanisms of therapeutic antibodies such as Rituximab.⁴⁷ Importantly, it has been that CD9 blockade could promote migration and inhibit adhesion of B-ALL cells to BM stroma, suggesting that CD9 antibody could disrupt leukemia-stroma interaction.

Interaction of leukemic cells with stromal cells in the BM microenvironment has been known to provide pro-survival and growth signals which could contribute to chemoresistance, minimal residual disease and relapse.⁴⁸ It has been well-documented that the BM stroma could render B-ALL cells resistant to chemotherapy-induced apoptosis.^{49,50} Such phenomenon, defined as cell adhesion-mediated drug resistance, is promoted in part by adhesion of leukemic cells to the BM stroma,⁵¹ which is controlled by binding of leukemia-expressing integrin VLA-4 with stromal cell-expressing VCAM-1 and other extracellular matrix components.⁵² These in vitro experiments provided direct evidence that CD9 was physically bound to integrin VLA-4 in B-ALL cells and thereby enhancing their affinity to VCAM-1. These data are in line with those reported in endothelial, ovarian and prostate cancer cells, which consistently showed that the association of CD9 with β1 subunit would result in integrin activation and enhancement of cellular adhesion.⁵³⁻⁵⁵ Other reported activities of CD9 in B-ALL cells included activation of Src and Rac1 to induce focal adhesion and rearrangement of actin cytoskeleton.^{14,15} Indeed, both Src-family kinases and Rac GTPase are known downstream intermediates of the integrin signaling.^{56,57} These data, together with others, collectively suggest that CD9 may act cooperatively with integrin VLA-4 to induce a signaling cascade that facilitates retention of leukemic blasts in the BM niche and thus, limiting chemosensitivity.

In summary, this study has provided data showing the prognostic impact of CD9 expression on predicting survival outcomes of pediatric B-ALL patients. In addition, the efficacy of targeting CD9 alone or in combination with chemotherapy for inhibition of B-ALL progression has been demonstrated. Significantly, this novel approach of CD9-targeted therapy was effective for subgroups of high-risk B-ALL. Mechanistically, evidence has been presented on the interaction of CD9 with the VLA-4/VCAM-1 axis and its roles in regulation of leukemia-stroma interaction and chemosensitivity. It is anticipated that CD9 antibody could be developed in adjunct to standard chemotherapy for treatment of high-risk or relapsed/refractory pediatric B-ALL, and potentially be extended for application in adult B-ALL and other malignancies that express the CD9 surface antigen.

All patents, patent applications, and other publications, including GenBank Accession Numbers, cited in this application are incorporated by reference in the entirety for all purposes.

SEQUENCE LISTING

SEQ ID NO: 1 Human CD9 mRNA sequence (GenBank Accession No. NM_001769.3,
1321 nucleotides)

| | |
|---|---|
| 1 | cttttcccgg cacatgcgca ccgcagcggg tcgcgcgccc taaggagtgg cactttttaa |
| 61 | aagtgcagcc ggagaccagc ctacagccgc ctgcatctgt atccagcgcc aggtcccgcc |
| 121 | agtcccagct gcgcgcgccc cccagtcccg cacccgttcg gcccaggcta agttagccct |
| 181 | caccatgccg gtcaaaggag gcaccaagtg catcaaatac ctgctgttcg gatttaactt |
| 241 | catcttctgg cttgccggga ttgctgtcct tgccattgga ctatggctcc gattcgactc |
| 301 | tcagaccaag agcatcttcg agcaagaaac taataataat aattccagct tctacacagg |
| 361 | agtctatatt ctgatcggag ccggcgccct catgatgctg gtgggcttcc tgggctgctg |
| 421 | cggggctgtg caggagtccc agtgcatgct gggactgttc ttcggcttcc tcttggtgat |
| 481 | attcgccatt gaaatagctg cggccatctg gggatattcc cacaaggatg aggtgattaa |
| 541 | ggaagtccag gagttttaca aggacaccta caacaagctg aaaaccaagg atgagcccca |
| 601 | gcgggaaacg ctgaaagcca tccactatgc gttgaactgc tgtggtttgg ctgggggcgt |
| 661 | ggaacagttt atctcagaca tctgccccaa gaaggacgta ctcgaaacct tcaccgtgaa |
| 721 | gtcctgtcct gatgccatca aagaggtctt cgacaataaa ttccacatca tcggcgcagt |
| 781 | gggcatcggc attgccgtgg tcatgatatt tggcatgatc ttcagtatga tcttgtgctg |
| 841 | tgctatccgc aggaaccgcg agatggtcta gagtcagctt acatccctga gcaggaaagt |
| 901 | ttacccatga agattggtgg gattttttgt ttgtttgttt tgttttgttt gttgtttgtt |
| 961 | gtttgttttt ttgccactaa ttttagtatt cattctgcat tgctagataa aagctgaagt |
| 1021 | tactttatgt ttgtctttta atgcttcatt caatattgac atttgtagtt gagcgggggg |
| 1081 | tttggtttgc tttggtttat attttttcag ttgtttgttt ttgcttgtta tattaagcag |
| 1141 | aaatcctgca atgaaaggta ctatatttgc tagactctag acaagatatt gtacataaaa |
| 1201 | gaattttttt gtctttaaat agatacaaat gtctatcaac tttaatcaag ttgtaactta |
| 1261 | tattgaagac aatttgatac ataataaaaa attatgacaa tgtcctggac tggtaaaaaa |
| 1321 | a |

SEQ ID NO: 2 Human CD9 protein sequence (GenBank Accession No.
NP_001760.1, 228 amino acids)
mpvkggtkci kyllfgfnfi fwlagiavla iglwlrfdsq tksifeqetn nnnssfytgv
yiligagalm mlvgflgccg avqesqcmlg lffgfllvif aieiaaaiwg yshkdevike
vqefykdtyn klktkdepqr etlkaihyal nccglaggve qfisdicpkk dvletftvks
cpdaikevfd nkfhiigavg igiavvmifg mifsmilcca irrnremv

### LIST OF REFERENCES

1. Pui CH, Mullighan CG, Evans WE, Relling MV. Pediatric acute lymphoblastic leukemia: where are we going and how do we get there? Blood. 2012;120(6):1165-1174.
2. Harrison C J. Cytogenetics of paediatric and adolescent acute lymphoblastic leukaemia. Br J Haematol. 2009; 144(2): 147-156.
3. Hunger S P, Mullighan C G. Acute Lymphoblastic Leukemia in Children. N Engl J Med. 2015;373(16):1541-1552.
4. Mullighan CG. Molecular genetics of B-precursor acute lymphoblastic leukemia. J Clin Invest. 2012;122(10):3407-3415.
5. Bhojwani D, Pui CH. Relapsed childhood acute lymphoblastic leukaemia. Lancet Oncol. 2013;14(6):e205-e217.
6. Kantarjian H, Thomas D, Wayne AS, O'Brien S. Monoclonal antibody-based therapies: a new dawn in the treatment of acute lymphoblastic leukemia. J Clin Oncol. 2012;30(31):3876-3883.
7. Armenian S H, Robison L L. Childhood cancer survivorship: an update on evolving paradigms for understanding pathogenesis and screening for therapy-related late effects. Curr Opin Pediatr. 2013;25(1):16-22.
8. Hemler M E. Tetraspanin functions and associated microdomains. Nat Rev Mol Cell Biol. 2005;6(10):801-811.
9. Yanez-Mo M, Barreiro O, Gordon-Alonso M, Sala-Valdes M, Sanchez-Madrid F. Tetraspanin-enriched microdomains: a functional unit in cell plasma membranes. Trends Cell Biol. 2009; 19(9):434-446.
10. Leung K T, Chan K Y, Ng P C, et al. The tetraspanin CD9 regulates migration, adhesion, and homing of human cord blood CD34+hematopoietic stem and progenitor cells. Blood. 2011;117(6):1840-1850.
11. Zoller M. Tetraspanins: push and pull in suppressing and promoting metastasis. Nat Rev Cancer. 2009;9(1):40-55.
12. Aoki Y, Watanabe T, Saito Y, et al. Identification of CD34+and CD34- leukemia-initiating cells in MLL-rearranged human acute lymphoblastic leukemia. Blood. 2015;125(6):967-980.
13. Nishida H, Yamazaki H, Yamada T, et al. CD9 correlates with cancer stem cell potentials in human B-acute lymphoblastic leukemia cells. Biochem Biophys Res Commun. 2009;382(1):57-62.
14. Yamazaki H, Xu CW, Naito M, et al. Regulation of cancer stem cell properties by CD9 in human B-acute lymphoblastic leukemia. Biochem Biophys Res Commun. 2011;409(1):14-21
15. Arnaud M P, Vallee A, Robert G, et al. CD9, a key actor in the dissemination of lymphoblastic leukemia, modulating CXCR4-mediated migration via RAC1 signaling. Blood. 2015;126(15):1802-1812.
16. Stary J, Zimmermann M, Campbell M, et al. Intensive chemotherapy for childhood acute lymphoblastic leukemia: results of the randomized intercontinental trial ALL IC-BFM 2002. J Clin Oncol. 2014;32(3):174-184.
17. Spiegel A, Kollet O, Peled A, et al. Unique SDF-1-induced activation of human precursor-B ALL cells as a result of altered CXCR4 expression and signaling. Blood. 2004; 103 (8):2900-2907.
18. Tanaka T, Kitamura F, Nagasaka Y, et al. Selective long-term elimination of natural killer cells in vivo by an anti-interleukin 2 receptor beta chain monoclonal antibody in mice. J Exp Med. 1993;178(3):1103-1107.
19. Hsieh Y T, Gang E J, Geng H, et al. Integrin alpha4 blockade sensitizes drug resistant pre-B acute lymphoblastic leukemia to chemotherapy. Blood. 2013; 121(10): 1814-1818.
20. Gang E J, Hsieh Y T, Pham J, et al. Small-molecule inhibition of CBP/catenin interactions eliminates drug-resistant clones in acute lymphoblastic leukemia. Oncogene. 2014;33(17):2169-2178.
21. Park E, Gang E J, Hsieh Y T, et al. Targeting survivin overcomes drug resistance in acute lymphoblastic leukemia. Blood. 2011;118(8):2191-2199.
22. Zhi Q, Wang Y, Wang X, et al. Predictive and prognostic value of preoperative serum tumor markers in resectable adenosqamous lung carcinoma. Oncotarget. 2016;7(40): 64798-64809.
23. Walter R B, Alonzo T A, Gerbing R B, et al. High expression of the very late antigen-4 integrin independently predicts reduced risk of relapse and improved outcome in pediatric acute myeloid leukemia: a report from the children's oncology group. J Clin Oncol. 2010;28(17):2831-2838.
24. Huang C L, Liu D, Masuya D, et al. MRP-1/CD9 gene transduction downregulates Wnt signal pathways. Oncogene. 2004;23(45):7475-7483.
25. Powner D, Kopp P M, Monkley S J, Critchley D R, Berditchevski F. Tetraspanin CD9 in cell migration. Biochem Soc Trans. 2011;39(2):563-567.
26. Desterke C, Martinaud C, Guerton B, et al. Tetraspanin CD9 participates in dysmegakaryopoiesis and stromal interactions in primary myelofibrosis. Haematologica. 2015; 100(6):757-767.
27. Reyes R, Monjas A, Yanez-Mo M, et al. Different states of integrin LFA-1 aggregation are controlled through its association with tetraspanin CD9. Biochim Biophys Acta. 2015;1853(10 Pt A):2464-2480.
28. Gustafson-Wagner E, Stipp C S. The CD9/CD81 tetraspanin complex and tetraspanin CD151 regulate alpha3betal integrin-dependent tumor cell behaviors by overlapping but distinct mechanisms. PLoS One. 2013;8(4):e61834.
29. Clappier E, Grardel N, Bakkus M, et al. IKZF1 deletion is an independent prognostic marker in childhood B-cell precursor acute lymphoblastic leukemia, and distinguishes patients benefiting from pulses during maintenance therapy: results of the EORTC Children's Leukemia Group study 58951. Leukemia. 2015;29(11):2154-2161.
30. Moricke A, Zimmermann M, Valsecchi M G, et al. Dexamethasone vs prednisone in induction treatment of pediatric ALL: results of the randomized trial AIEOP-BFM ALL 2000. Blood. 2016;127(17):2101-2112.
31. Kischel P, Bellahcene A, Deux B, et al. Overexpression of CD9 in human breast cancer cells promotes the development of bone metastases. Anticancer Res. 2012;32(12):5211-5220.
32. Hori H, Yano S, Koufuji K, Takeda J, Shirouzu K. CD9 expression in gastric cancer and its significance. J Surg Res. 2004;117(2):208-215.
33. Kohmo S, Kijima T, Otani Y, et al. Cell surface tetraspanin CD9 mediates chemoresistance in small cell lung cancer. Cancer Res. 2010;70(20):8025-8035.
34. Sauer G, Windisch J, Kurzeder C, et al. Progression of cervical carcinomas is associated with down-regulation of CD9 but strong local re-expression at sites of transendothelial invasion. Clin Cancer Res. 2003;9(17): 6426-6431.
35. Wang J C, Begin L R, Berube N G, et al. Down-regulation of CD9 expression during prostate carcinoma progression is associated with CD9 mRNA modifications. Clin Cancer Res. 2007;13(8):2354-2361.
36. Tang M, Yin G, Wang F, et al. Downregulation of CD9 promotes pancreatic cancer growth and metastasis through upregulation of epidermal growth factor on the cell surface. Oncol Rep. 2015;34(1):350-358.
37. De Bruyne E, Bos TJ, Asosingh K, et al. Epigenetic silencing of the tetraspanin CD9 during disease progression in multiple myeloma cells and correlation with survival. Clin Cancer Res. 2008;14(10):2918-2926.
38. Hemler ME. Tetraspanin proteins promote multiple cancer stages. Nat Rev Cancer. 2014;14(1):49-60.
39. Wang H X, Sharma C, Knoblich K, Granter S R, Hemler M E. EWI-2 negatively regulates TGF-beta signaling leading to altered melanoma growth and metastasis. Cell Res. 2015;25(3):370-385.
40. Herr M J, Kotha J, Hagedorn N, Smith B, Jennings L K. Tetraspanin CD9 promotes the invasive phenotype of human fibrosarcoma cells via upregulation of matrix metalloproteinase-9. PLoS One. 2013;8(6):e67766.
41. Sanjuan-Pla A, Bueno C, Prieto C, et al. Revisiting the biology of infant t(4;11)/MLL-AF4+B-cell acute lymphoblastic leukemia. Blood. 2015;126(25):2676-2685.
42. Leonard J T, Rowley J S, Eide C A, et al. Targeting BCL-2 and ABL/LYN in Philadelphia chromosome-positive acute lymphoblastic leukemia. Sci Transl Med. 2016;8(354):354ra114.
43. Hwang J R, Jo K, Lee Y, et al. Upregulation of CD9 in ovarian cancer is related to the induction of TNF-alpha gene expression and constitutive NF-kappaB activation. Carcinogenesis. 2012;33(1):77-83.
44. Nakamoto T, Murayama Y, Oritani K, et al. A novel therapeutic strategy with anti-CD9 antibody in gastric cancers. J Gastroenterol. 2009;44(9):889-896.
45. Ovalle S, Gutierrez-Lopez M D, Olmo N, et al. The tetraspanin CD9 inhibits the proliferation and tumorigenicity of human colon carcinoma cells. Int J Cancer. 2007;121(10):2140-2152.
46. Murayama Y, Miyagawa J, Oritani K, et al. CD9-mediated activation of the p46 Shc isoform leads to apoptosis in cancer cells. J Cell Sci. 2004;117(Pt 15):3379-3388.
47. Scott A M, Wolchok J D, Old L J. Antibody therapy of cancer. Nat Rev Cancer. 2012;12(4):278-287.
48. Lane S W, Scadden D T, Gilliland D G. The leukemic stem cell niche: current concepts and therapeutic opportunities. Blood. 2009; 114(6): 1150-1157.
49. Iwamoto S, Mihara K, Downing J R, Pui C H, Campana D. Mesenchymal cells regulate the response of acute lymphoblastic leukemia cells to asparaginase. J Clin Invest. 2007; 117(4):1049-1057.
50. Tesfai Y, Ford J, Carter K W, et al. Interactions between acute lymphoblastic leukemia and bone marrow stromal cells influence response to therapy. Leuk Res. 2012;36(3):299-306.
51. Meads M B, Gatenby R A, Dalton W S. Environment-mediated drug resistance: a major contributor to minimal residual disease. Nat Rev Cancer. 2009;9(9):665-674.
52. Jacamo R, Chen Y, Wang Z, et al. Reciprocal leukemia-stroma VCAM-1/VLA-4-dependent activation of NF-kappaB mediates chemoresistance. Blood. 2014; 123 (17):2691-2702.
53. Gutierrez-Lopez M D, Ovalle S, Yanez-Mo M, et al. A functionally relevant conformational epitope on the CD9 tetraspanin depends on the association with activated betal integrin. J Biol Chem. 2003;278(1):208-218.
54. Kotha J, Longhurst C, Appling W, Jennings L K. Tetraspanin CD9 regulates beta 1 integrin activation and enhances cell motility to fibronectin via a PI-3 kinase-dependent pathway. Exp Cell Res. 2008;314(8):1811-1822.
55. Pellinen T, Rantala J K, Arjonen A, et al. A functional genetic screen reveals new regulators of betal-integrin activity. J Cell Sci. 2012; 125(Pt 3):649-661.
56. Shattil S J. Integrins and Src: dynamic duo of adhesion signaling. Trends Cell Biol. 2005;15(8):399-403.
57. Akhtar N, Streuli C H. Rac1 links integrin-mediated adhesion to the control of lactational differentiation in mammary epithelia. J Cell Biol. 2006;173(5):781-793.

What is claimed is:

1. A method for assessing risk for a blood cancer in a subject, comprising the steps of:
(a) measuring CD9 level in a sample comprising lymphoblasts taken from the subject,
(b) comparing the CD9 level obtained in step (a) with a standard control, and
(c) determining the subject, who has an increased CD9 level compared with the standard control, as having an increased risk for a blood cancer.

2. The method of claim 1, wherein the blood cancer is acute lymphoblastic leukemia (ALL).

3. The method of claim 1, wherein the CD9 level is CD9 protein level.

4. The method of claim 1, wherein the CD9 level is CD9 mRNA level.

5. The method of claim 3, wherein step (a) comprises an immunoassay using an antibody that specifically binds the CD9 protein.

6. The method of claim 3, wherein step (a) comprises flow cytometry.

7. The method of claim 4, wherein step (a) comprises a polynucleotide amplification reaction.

8. The method of claim 7, wherein the amplification reaction is a polymerase chain reaction (PCR).

9. The method of claim 8, wherein the PCR is a reverse transcriptase-PCR (RT-PCR).

10. The method of claim 4, wherein step (a) comprises a polynucleotide hybridization assay.

11. The method of claim 10, wherein the polynucleotide hybridization assay is a Southern Blot analysis or Northern Blot analysis or an in situ hybridization assay.

12. The method of claim 1, when the subject is indicated as having an increased risk for blood cancer, further comprising repeating step (a) at a later time using the sample type of sample from the subject, wherein an increase in the CD9 level at the later time as compared to the amount from the original step (a) indicates a heightened risk of blood cancer, and a decrease indicates a lessened risk for blood cancer.

13. The method of claim 1, when the subject is indicated as having an increased risk for blood cancer, further comprising a step of administering to the subject an inhibitor of CD9.

14. The method of claim 13, wherein the blood cancer is ALL and the inhibitor is a neutralizing antibody against CD9, and optionally the patient is receiving chemotherapy at the same time.

15. A method for assessing likelihood of recurrence of a blood cancer or death due to the blood cancer in a patient suffering from the blood cancer, comprising the steps of:
(a) measuring CD9 level in a sample comprising lymphoblasts taken from the patient,
(b) comparing the CD9 level obtained in step (a) with CD9 level from another sample of the same type obtained from a second patient also suffering from the blood cancer and measured by step (a), and
(c) determining the patient, who has a higher CD9 level compared with the CD9 level determined in the other sample of the same type obtained from a second patient and measured by step (a), as having an increased risk for recurrence of the blood cancer or death due to the blood cancer compared with the second patient.

16. The method of claim 15, wherein the blood cancer is ALL.

17. The method of claim 15, wherein the CD9 level is CD9 protein level.

18. The method of claim 15, wherein step (a) comprises an immunoassay using an antibody that specifically binds the CD9 protein.

19. The method of claim 15, wherein step (a) comprises flow cytometry.

20. The method of claim 15, wherein the CD9 level is CD9 mRNA level.

21. The method of claim 20, wherein step (a) comprises a polynucleotide amplification reaction.

22. The method of claim 21, wherein the amplification reaction is a polymerase chain reaction (PCR).

23. The method of claim 22, wherein the PCR is a reverse transcriptase-PCR (RT-PCR).

24. The method of claim 20, wherein step (a) comprises a polynucleotide hybridization reaction.

25. The method of claim 15, wherein the patient has previously received treatment for the blood cancer.

26. The method of claim 15, wherein the second patient has an CD9 level essentially the same as the CD9 level determined in another sample of the same type that is (1) obtained from a healthy individual who does not have the blood cancer or an elevated risk for developing the blood cancer; and (2) measured by step (a).

27. A kit for detecting or characterizing a blood cancer in a subject, comprising (1) a standard control that provides an average amount of CD9 protein or CD9 mRNA in lymphoblasts; and (2) an agent that specifically and quantitatively identifies CD9 protein or CD9 mRNA.

28. The kit of claim 27, wherein the agent is an antibody that specifically binds the CD9 protein.

29. The kit of claim 27, wherein the agent is a polynucleotide probe that hybridizes with CD9 mRNA.

30. The kit of claim 27, wherein the agent comprises a detectable moiety.

31. The kit of claim 27, further comprising an instruction manual.

32. A method for inhibiting growth of a CD9+ lymphoblast cell, comprising contacting the cell with an effective amount of a neutralizing antibody against CD9 protein or a nucleic acid encoding a polynucleotide sequence that is complementary to at least a segment of CD9 mRNA and suppresses CD9 mRNA expression.

33. The method of claim 32, wherein the nucleic acid comprises a promoter directing the transcription of the polynucleotide sequence.

34. The method of claim 33, wherein the promoter is a lymphoblast-specific promoter.

35. The method of claim 32, wherein the nucleic acid encodes an antisense RNA, miRNA, or siRNA.

36. The method of claim 32, wherein the CD9+ lymphoblast cell is within a patient's body.

37. The method of claim 36, when the patient is receiving chemotherapy and a neutralizing antibody against CD9.


SEQUENCE LISTING (4 sequences)

SEQ ID NO: 1 - DNA, 1321 bp, Homo Sapiens
cttttcccggcacatgcgcaccgcagcgggtcgcgcgccctaaggagtggcactttttaaaagtgcagccggagaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctggactggtaaaaaaa

SEQ ID NO: 2 - protein, 228 aa, Homo Sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 3 - DNA, 20 bp, Artificial Sequence
  Synthetic Construct
gggatattcccacaaggatg

SEQ ID NO: 4 - DNA, 20 bp, Artificial
  Synthetic Construct
gcactcacatcgctacatca